(19) United States
(12) Patent Application Publication
(10) Pub. No.: US 2025/0037788 A1
(43) Pub. Date: Jan. 30, 2025

(54) Methods and Systems for Learning Gene Regulatory Networks Using Sparse Gaussian Mixture Models

(71) Applicant: The Board of Trustees of the Leland Stanford Junior University, Stanford, CA (US)

(72) Inventors: Shaimaa Hesham Bakr, Stanford, CA (US); Olivier Gevaert, Palo Alto, CA (US)

(73) Assignee: The Board of Trustees of the Leland Stanford Junior University, Stanford, CA (US)

(21) Appl. No.: 18/712,661

(22) PCT Filed: Nov. 22, 2022
(86) PCT No.: PCT/US2022/080366

§ 371 Date: May 22, 2024

Related U.S. Application Data
(60) Provisional application No. 63/264,508, filed on Nov. 23, 2021.

(51) Int. Cl. G16B 5/00; G06F 30/27; G16B 25/10

(52) U.S. Cl. CPC G16B 5/00; G06F 30/27; G16B 25/10

(57) ABSTRACT

Methods and systems for constructing gene modules with regulator genes and target genes are provided. A Gaussian mixed model can construct gene modules using RNA sequencing data. Sets of gene modules can be compared to identify shared or unique biological processes.

### CROSS REFERENCE TO RELATED APPLICATIONS

This application claims priority to U.S. Provisional Application Ser. No. 63/264,508, entitled “Methods and Systems for Learning Gene Regulatory Networks Using Sparse Gaussian Mixture Models,” filed Nov. 23, 2021, which is incorporated herein by reference in its entirety.

### STATEMENT REGARDING FEDERALLY SPONSORED RESEARCH OR DEVELOPMENT

This invention was made with Government support under contracts CA199241, CA217851, and CA260271 awarded by the National Institutes of Health. The Government has certain rights in the invention.

### TECHNOLOGICAL FIELD

The disclosure is generally directed to methods and systems to generate statistical computational models, including Gaussian mixture models, for learning gene regulatory networks, which can be utilized in drug discovery.

### BACKGROUND

Gene regulatory networks (GRNs) are one class of tools that can be applied to genomic data to improve the understanding of systems biology and to uncover the molecular basis of disease. Network methods can be used to model gene-level relationships, protein-protein and cell-cell interactions. Several approaches to learning GRNs exist including graph and module-based methods. In graph methods, a graph is created based on the expression data and then the graph is analyzed to extract subnetworks, with hub genes assumed to be regulators of target genes in these subnetworks. Hub genes are a subset of highly connected genes, relative to the other, less connected, downstream targets. Such scale-free network structure mimics the nature of biological networks. Module-based methods typically cluster co-expressed genes directly into gene modules and as a second step identify regulators of these gene modules. Example of module-based methods include CONEXIC, AMARETTO and CaMoDi, which have been shown to be more robust and better recapitulate underlying biology than graph-based methods. AMARETTO is a module-based tool that clusters co-expressed genes and assigns each module to its regulators using sparse linear regression. AMARETTO outperforms other module methods in its ability to leverage information from copy number variation and methylation data to improve the discovery of regulators and their assignment to gene modules. The genomic and epigenetic events inform the choice of candidate drive genes, which are used then as features selected by sparse linear regression (e.g., LASSO). The resulting modules are functionally annotated using Gene Set Enrichment Analysis (GSEA) techniques, elucidating the role of driver genes in cancer development and progression.

### SUMMARY

Several embodiments are directed to methods and systems of constructing gene modules. In many embodiments, a Gaussian mixed model is utilized to construct a set of gene modules. In several embodiments, the Gaussian mixed model is combined with norm regularization yielding a sparse Gaussian mixed model. In many embodiments, sets of gene modules are compared to identify shared or unique biological processes within each set of gene modules. In several embodiments, regulator genes and target genes within each gene module are identified. In some embodiments, drug targets within one or more gene modules are identified.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow diagram of a method to build gene modules with a Gaussian mixture model in accordance with various embodiments.

FIG. 2 provides an example of utilizing RNA sequencing data to yield cancer gene modules in accordance with various embodiments.

FIG. 3 provides a flow diagram of a method to build gene modules with a Gaussian mixture model in accordance with various embodiments.

FIG. 4 provides a conceptual illustration of a computational processing system in accordance with various embodiments.

FIGS. 5A and 5B provides data graphs depicting the performance of SparseGMM and AMARETTO using the TCGA HCC data (FIG. 5A) and GTEx data (FIG. 5B), generated in accordance with various embodiments. Robustness of clustering is evaluated using adjusted Rand index. Validation of regulators is represented by R-squared. Degree of sparsity is evaluated using statistics on the number of drivers. Module size informs the choice of regularization parameter value.

FIG. 5C provides a table comparing SparseGMM to AMARETTO at different regularization values: 50, 500 and 5000, generated in accordance with various embodiments. Robustness of clustering is evaluated using adjusted Rand index. Validation of regulators is represented by R-squared. Degree of sparsity is evaluated using statistics on the number of drivers. Module size informs the choice of regularization parameter value.

FIGS. 6A and 6B provide data graphs depicting the performance of SparseGMM at different regularization values, comparing TCGA LUAD data (FIG. 6A) and TCGA HNSC data (FIG. 6B), generated in accordance with various embodiments. Robustness of clustering is evaluated using adjusted Rand index. Validation of regulators is represented by R-squared. Degree of sparsity is evaluated using statistics on the number of drivers. Module size informs the choice of regularization parameter value.

FIG. 7 provides a representation of the SparseGMM module network, generated in accordance with various embodiments. Left: A sample module network obtained through community detection algorithm to cancer and normal liver modules, after running SparseGMM with different initializations. Right: The community detection clusters robust modules together into distinct subnetworks. Subnetworks at the periphery represent robust modules. Subnetworks are then functionally annotated using gene set enrichments analysis applied to MSigDB gene sets. Highlighted here are robust modules from normal liver, liver cancer, as well as shared communities that contain modules occurring in normal and cancer tissue.

FIG. 8 provides a table reporting ReMap validated regulators of robust normal liver and liver cancer communities, generated in accordance with various embodiments. Robust communities were defined by having Jaccard Index>=0.7. Main pathway of each community was revealed through gene set enrichment analysis of SparseGMM modules in GTEx and TCGA data against MSigDB collections. Validation of regulators is established with an adjusted p-value <0.05.

FIG. 9 provides a table reporting LINC validated regulators of robust normal liver and liver cancer communities, generated in accordance with various embodiments. Robust communities were defined by having Jaccard Index>=0.7. Main pathway of each community was revealed through gene set enrichment analysis of SparseGMM modules in GTEx and TCGA data against MSigDB collections. Validation of regulators is established with an adjusted p-value <0.05.

FIGS. 10A to 10C provide cell type identification based on Pangloa DB markers, comparing original data annotation and Seurat-based clustering, generated in accordance with various embodiments. FIG. 10A provides average expression of different cell type Pangloa DB markers. FIG. 10B provides original cell type assignments (left panel) and Seurat clusters (right panel). FIG. 10C provides cell type specific expression of communities 21 (dendritic cells) and 60 (T cells).

FIGS. 11A and 11B provides single cell evaluation of highly robust communities, generated in accordance with various embodiments. FIG. 11A provides average expression and gene number of T cell community and myeloid community cell types. FIG. 11B provides average expression and gene number of cell cycle community cell types. FIG. 11B also provides cell type annotation and most significant gene set enrichments for the three communities.

FIGS. 12A and 12B provides single cell evaluation of highly robust communities, generated in accordance with various embodiments. FIGS. 12A and 12B provide expression of target genes in T cell communities, myeloid communities and cell cycle communities. FIG. 12B also provides cell type assignment.

FIG. 12C provides a boxplot of average expression of cell cycle community target genes in blood, normal and cancer immune cells, generated in accordance with various embodiments.

FIG. 13 provides a boxplot of cell cycle phase versus expression of cell cycle community target genes, generated in accordance with various embodiments. Higher expression of cell cycle genes corresponds to proliferative G2M and S phases.

FIGS. 14 to 16 provide analysis of high entropy genes, generated in accordance with various embodiments. FIG. 14 provides a boxplot showing difference in mean entropy distribution for high entropy target genes in GTEX and TCGA, reflecting heterogeneity of cancer samples. Entropy is calculated from the posterior probability of target genes in each data set and the mean is calculated over several runs of SparseGMM on each data set. FIG. 15 provides a graph showing distribution of communities of high entropy genes. FIG. 16 provides expression of communities with high entropy genes.

### DETAILED DESCRIPTION

Turning now to the drawings and data, various methods and systems for learning gene regulatory networks utilizing statistical computational models are described, in accordance with the various embodiments of the description. In several embodiments, a Bayesian generative model is built and utilized to learn regulatory relationships between genes. In many embodiments, to better understand the relationships among genes, some genes are classified as regulators and some genes are classified targets of regulators. In this context, regulator genes are genes undergoing genomic events that are relevant to a biological process (e.g., cancer progression or tumor growth). Further in this context, target genes are genes whose expression is controlled by the regulator genes, and which contribute to the relevant biological process. In several embodiments, the Bayesian generative model incorporates a Gaussian mixtures model. In many embodiments, the Bayesian generative model incorporates norm regularization, which can be combined with the Gaussian mixtures model to enforce sparsity on the regulator weights. In several embodiments, an expectation-maximization (EM)-based algorithm is developed, which can be utilized to obtain a maximum a posteriori (MAP) estimate of the Gaussian mixture of parameters.

In accordance with several embodiments, a gene module analysis model was developed using a Bayesian framework, whereby the clustering of target genes and the assignment of regulators are combined in one step, which allows genes to be associated to multiple modules simultaneously. In many embodiments, a confidence interval can be calculated for an assignment of a regulator to its modules. More specifically, for several embodiments, a sparse Gaussian mixture model (GMM) inference is utilized, where the mixture mean is represented as a weighted sparse vector of regulator expression level. This novel framework overcomes a major limitation in module-based methods by allowing probabilistic assignments of target genes to modules and significance estimates of individual regulator coefficients. Utilizing of a GMM improved performance in sparsity, compared to previous gene networking methods, choosing fewer genes as true regulators, and confirming biological knowledge of the scale-free nature of gene networks.

To test this model, it was applied to GTEx data from healthy liver tissue, as well as hepatocellular carcinoma (HCC) samples from TCGA (see Exemplary Embodiments). The model is able to successfully recover healthy tissue modules such as energy metabolism pathways and cancer specific modules involved in antigen presentation, immune response and blood coagulation. Common modules in healthy liver and hepatocellular carcinoma were also discovered, such as modules for inflammation and steroid biosynthesis, among others. Further, single cell data set of CD45+ immune cells was used to evaluate immune related modules discovered using the bulk sequencing data. The single cell evaluation of immune modules was able to decouple distinct myeloid and lymphoid biological processes in HCC micro-environment. The results demonstrate the ability of these methods to represent GRNs as potentially overlapping gene modules as demonstrated on bulk and single cell RNA seq data.

Further, contrary to previous methods, the probabilistic assignment approach taken by a Gaussian mixture model (especially one that is sparse) is potentially superior for modeling genes with multiple biological functions. Thus, in many embodiments, the entropy of a gene was defined to be the entropy of the estimated module-assignment probability, and show that it can then be used as an indicator of a multifunctional biological role based on joint membership to two or more modules. These multifunctional genes could in turn translate to multifunctional proteins having central roles in the crosstalk between two or more pathways in cancer cells, and, thus become attractive targets for overcoming drug resistance through compensation mechanisms. It was shown that high-entropy genes are more common in cancer samples than in healthy tissue, and these were associated to crosstalk between several pathways including TP53, interferon gamma and TNF alpha. The analysis of high entropy genes exemplifies ways in which major cancer pathways share key multifunctional components.

### Learning Gene Regulatory Relationships

Several embodiments are directed to learning gene regulatory relationships. In many embodiments, a Bayesian model is utilized to construct gene regulatory networks. In several embodiments, the Bayesian model incorporates a Gaussian mixture model. In many embodiments, the Gaussian mixture model is combined with norm regularization (e.g., L1-norm regularization), which can prevent over fitting and provide a sparse solution. In many embodiments, a maximum a posteriori (MAP) of the Gaussian mixture of parameters is estimated.

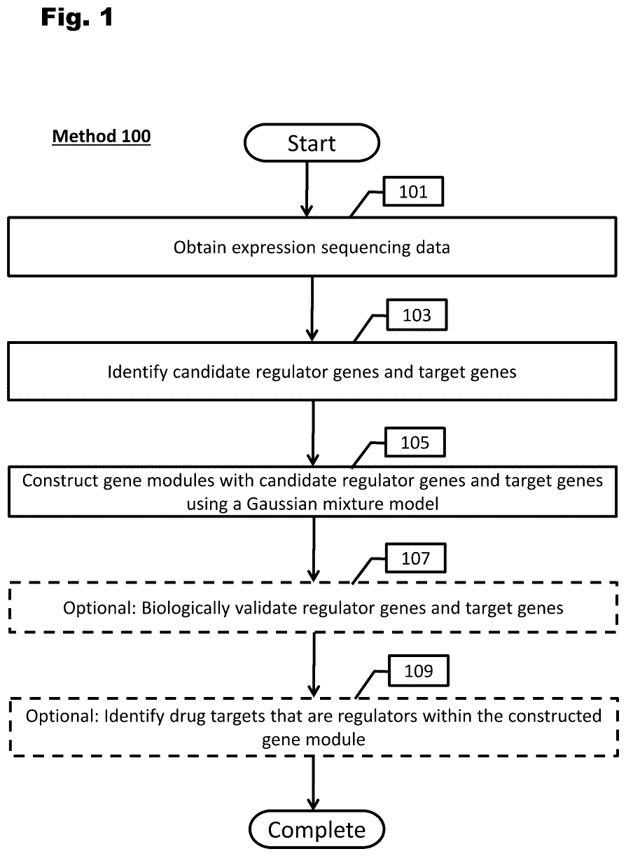

Provided in FIG. 1 is a method to construct gene modules using a Gaussian mixture model in accordance with various embodiments. Method 100 begins with obtaining (101) expression sequencing data of one or more biological samples. Expression sequencing data can be obtained by any appropriate method that can directly measure gene expression or infer gene expression. In some embodiments, RNA molecules are extracted from biological sample and prepped for sequencing. Any method of sequencing can be utilized. In many embodiments, high throughput sequencing is performed utilizing a sequencer, such as ones manufactured by Illumina. Further, a biological sample can be any sample with expressing RNA or containing RNA molecules. Biological samples include (but are not limited to) in vivo samples, in vitro samples, animal tissue, animal biopsy, tumor biopsy, bodily fluids (e.g., blood), cell culture, a single cell, healthy samples, and samples of a medical disorder. In some instances, a biological sample is extracted from a patient having a medical disorder. In some instances, the patient has a cancer or other neoplastic growth.

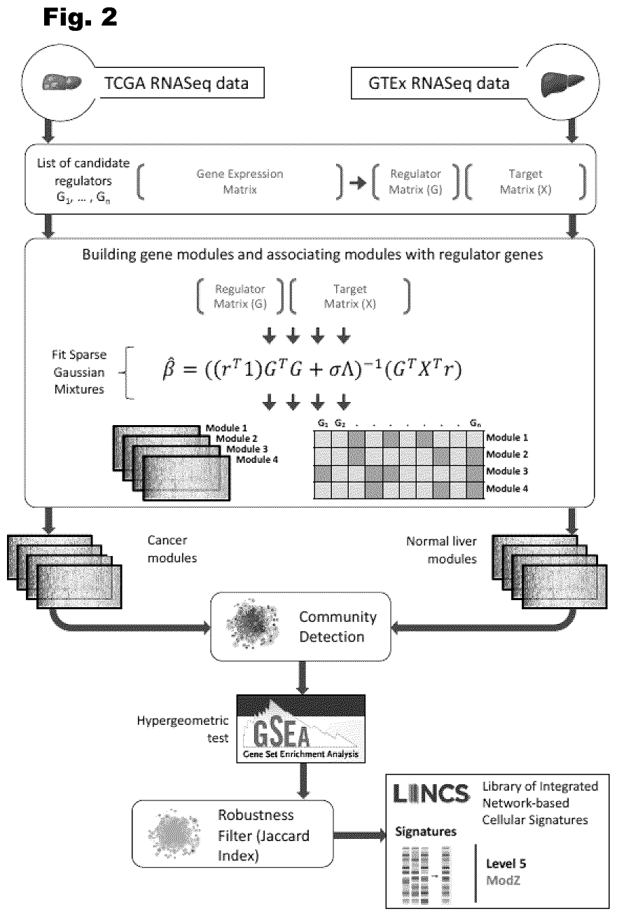

Method 100 further identifies (103) gene regulators and gene targets utilizing a sparse Gaussian mixture model (GMM). In many embodiments, the GMM incorporates norm regularization (e.g., L1-norm regularization). Provided in the Exemplary Embodiments is an example of how to construct a sparse GMM utilizing L1-norm regularization. Generally, a GMM utilizes a gene expression matrix to identify matrices of regulator genes and matrices of target genes (FIG. 2). In this context, regulator genes are genes undergoing genomic events that are relevant to one or more biological processes of the biological sample. Target genes are genes whose expression is controlled by regulator genes, and which contribute to the one or more biological processes of the biological sample. In some embodiments, the biological sample is a cancer, and identified regulator genes can be drivers (or repressors) of cancer progression, and identified target genes are acted upon by the regulator genes to promote the cancer progression.

Method 100 also constructs (105) gene modules with candidate regulator genes and target genes using regulator matrices, the target matrices, and the GMM. In several embodiments, the sparse GMM is defined as follows:

$$\hat{β} = {( ( {τ}^{T} 1 ) {G}^{T} G + σ Λ )}^{- 1} ( {G}^{T} {X}^{T} τ )$$

where G are the regulator genes, X are the target genes (FIG. 2). Numerous gene modules can be generated, each gene module being a biological process occurring in the biological sample. Further, within each gene within the module is labeled as a gene regulator or gene target (see FIG. 2).

In several embodiments, communities of modules are identified. A community can be defined by the average pairwise Jaccard Index between two or more modules. Further, in many embodiments, a biological function can be assigned to an individual module or a community.

Method 100 also optionally biologically validates (107) the regulator genes and target genes of one or more modules. Accordingly, biological experimentation can be performed to confirm that a regulator does in fact regulate the target genes within its module. To do so, a genetic and/or biochemical experiment can be performed that modulates expression or function of one or more regulators; the effect on target gene expression can be assessed.

Furthermore, Method 100 can optionally identify (109) drug targets within the constructed gene module. In many embodiments, a drug target is a regulator of the gene module. Further, preclinical assessment can be performed to assess compounds (e.g., small molecules, biologics, medicinals) that can modulate a regulator. In some instances, an assay that assesses regulator function is developed and performed. To perform the assay, a set of one or more compounds are individually (or in various combinations) applied to a sample in which regulator is assessable, and the effect of the compound on regulator function is assessed. A sample can be biological cell, tissue, lysates, isolated proteins, or any sample in which the regulator can perform its function and modulation of that function can be assessed.

While specific examples of processes for constructing a gene module utilizing Gaussian mixture models are described above, one of ordinary skill in the art can appreciate that various steps of the process can be performed in different orders and that certain steps may be optional according to some embodiments of the disclosure. As such, it should be clear that the various steps of the process could be used as appropriate to the requirements of specific applications. Furthermore, any of a variety of processes for constructing a gene module appropriate to the requirements of a given application can be utilized in accordance with various embodiments of the disclosure.

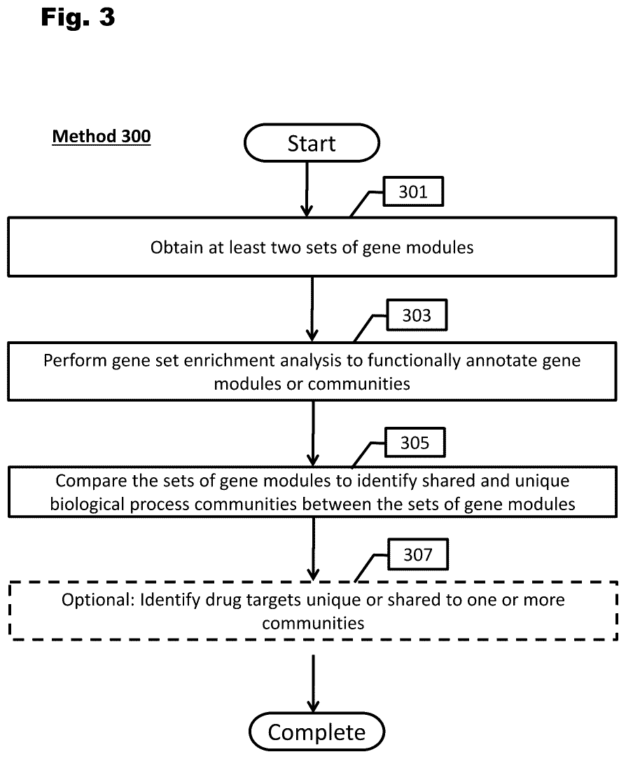

Provided in FIG. 3 is method to identify shared and unique biological process communities between two or more sets of gene modules. In accordance with many embodiments, this method can be utilized to identify shared and/or unique biological processes between two or more samples that have had their gene regulatory network analyzed.

Method 300 obtains (301) at least two sets of gene modules. In many embodiments, the gene modules have been constructed using a GMM, such as (for example) the embodiments described in FIG. 1 or in the Exemplary Embodiments. In several embodiments, each set of gene modules corresponds with a particular biological sample. In many embodiments, gene modules are constructed for two or more biological samples to be compared and analyzed. In some embodiments, the two or more biological samples comprise a reference or control (e.g., healthy sample) sample and an experimental sample (e.g., sample derived from a medical condition). In some particular embodiments, the two or more biological samples comprise a sample derived from a cancer and a healthy sample.

In several embodiments, communities of modules are identified. A community can be defined by the average pairwise Jaccard Index between two or more modules.

Method 300 also performs (303) gene set enrichment analysis (GSEA) to functionally annotate gene modules or communities. GSEA can be performed by any appropriate means. In some embodiments, GSEA is applied using MSigDB collections. In some embodiments, a GSEA result is filtered via a statistical method to enrich the result.

Method 300 further compares (305) the sets of gene modules to identify shared and unique biological process communities between the sets of gene modules. In several embodiments, identified communities that are present within each set of modules is shared. In many embodiments, identified communities that are present within one set of modules but not the other(s) is unique. Accordingly, comparison of the sets of gene modules can identify biological processes that are shared or unique within the biological samples. For example, when comparing a healthy biological sample with a biological sample derived from a medical condition, unique biological processes within or absent in the medical condition sample can be identified. Unique biological processes may relate to the pathology of the medical sample.

Furthermore, Method 300 can optionally identify (307) drug targets within the unique or shared communities. In many embodiments, a drug target is a regulator of the biological process of a community. Further, preclinical assessment can be performed to assess compounds (e.g., small molecules, biologics, medicinals) that can modulate a regulator. In some instances, an assay that assesses regulator function is developed and performed. To perform the assay, a set of one or more compounds are individually (or in various combinations) applied to a sample in which regulator is assessable, and the effect of the compound on regulator function is assessed. A sample can be biological cell, tissue, lysates, isolated proteins, or any sample in which the regulator can perform its function and modulation of that function can be assessed.

While specific examples of processes for comparing gene modules to identify shared and unique processes are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for comparing gene modules appropriate to the requirements of a given application can be utilized in accordance with various embodiments of the invention.

### Computational Processing System

A computational processing system to construct and/or compare gene modules in accordance with various embodiments of the disclosure typically utilizes a processing system including one or more of a CPU, GPU and/or other processing engine. In some embodiments, the computational processing system is housed within a computing device. In certain embodiments, the computational processing system is implemented as a software application on a computing device such as (but not limited to) mobile phone, a tablet computer, a wearable device (e.g., watch and/or AR glasses), and/or portable computer.

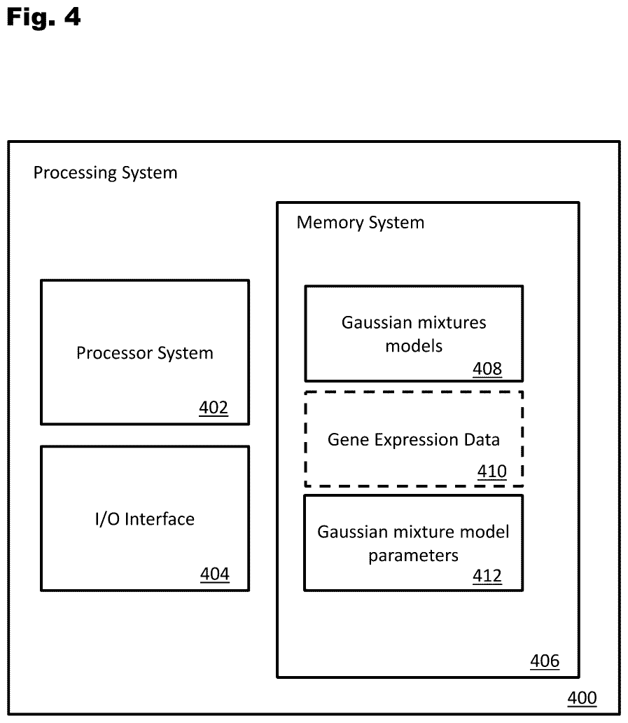

A computational processing system in accordance with various embodiments of the disclosure is illustrated in FIG. 4. The computational processing system 400 includes a processor system 402, an I/O interface 404, and a memory system 406. As can readily be appreciated, the processor system 402, I/O interface 404, and memory system 406 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing a Gaussian mixture model application 408, which can be combined with a norm regularization. The Gaussian mixture model application can be downloaded and/or stored in non-volatile memory. When executed the Gaussian mixture model application is capable of configuring the processing system to implement computational processes including (but not limited to) the computational processes described above and/or combinations and/or modified versions of the computational processes described above. In several embodiments, the Gaussian mixture model application 408 utilizes gene expression data 410, which can optionally be stored in the memory system, to perform gene module construction. In certain embodiments, the Gaussian mixture model application 408 utilizes model parameters 412 stored in memory to process RNA sequencing data using GMM to perform processes including (but not limited to) constructing and/or comparing gene modules. Model parameters 412 for any of a variety of GMM including (but not limited to) the various models described above. In several embodiments, the RNA sequencing data 410 is temporarily stored in the memory system during processing and/or saved for use in training/retraining of model parameters.

While specific computational processing systems are described above with reference to FIG. 4, it should be readily appreciated that computational processes and/or other processes utilized in the provision of gene module construction and uses thereof in accordance with various embodiments of the disclosure can be implemented on any of a variety of processing devices including combinations of processing devices. Accordingly, computational devices in accordance with embodiments of the disclosure should be understood as not limited to specific computational processing systems and/or gene module construction systems. Computational devices can be implemented using any of the combinations of systems described herein and/or modified versions of the systems described herein to perform the processes, combinations of processes, and/or modified versions of the processes described herein.

### Exemplary Embodiments

The embodiments of the disclosure will be better understood with the various examples provided within. Provided within is a manuscript and supplements to provide examples of performing the various embodiments as described.

Here, a new method is presented, SparseGMM, which uses a Bayesian latent variable approach to model the relationship between regulators and downstream target genes (see Methods, Supplementary Note 2). To validate this approach, the method was applied to two bulk gene expression liver data sets of normal liver and liver. Gene modules in normal tissue were constructed using publicly available data from GTEx project, while cancer modules were constructed using hepatocellular carcinoma data from the TCGA project (FIG. 2). Community detection methods were used to screen gene modules for robustness and to uncover shared biology between normal liver tissue and liver cancer. Next, these communities were evaluated in an independent single cell data set containing CD45+ immune cells from HCC cancer patients and analyzed the expression of these communities in different immune cell populations.

### Results

Technical validation of SparseGMM

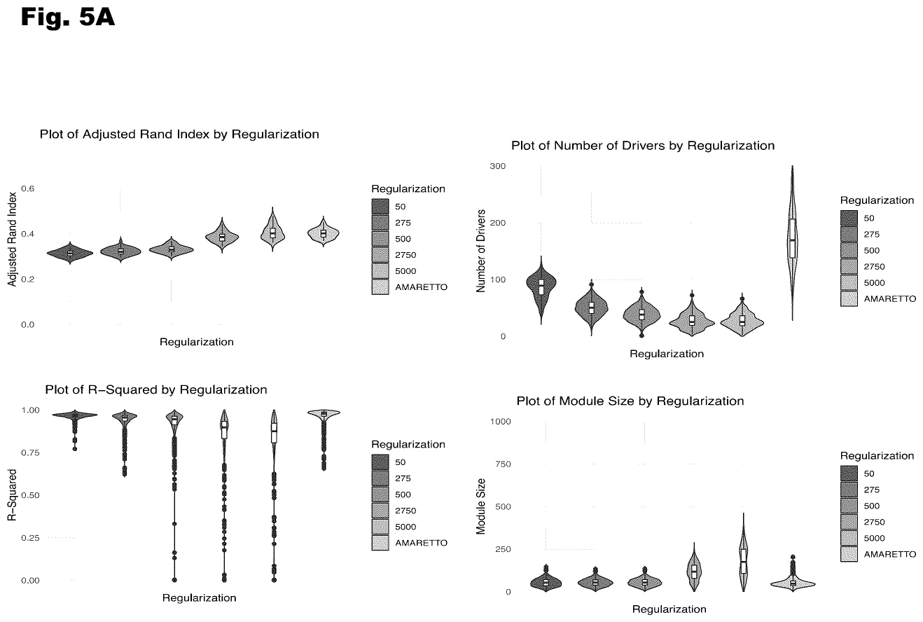

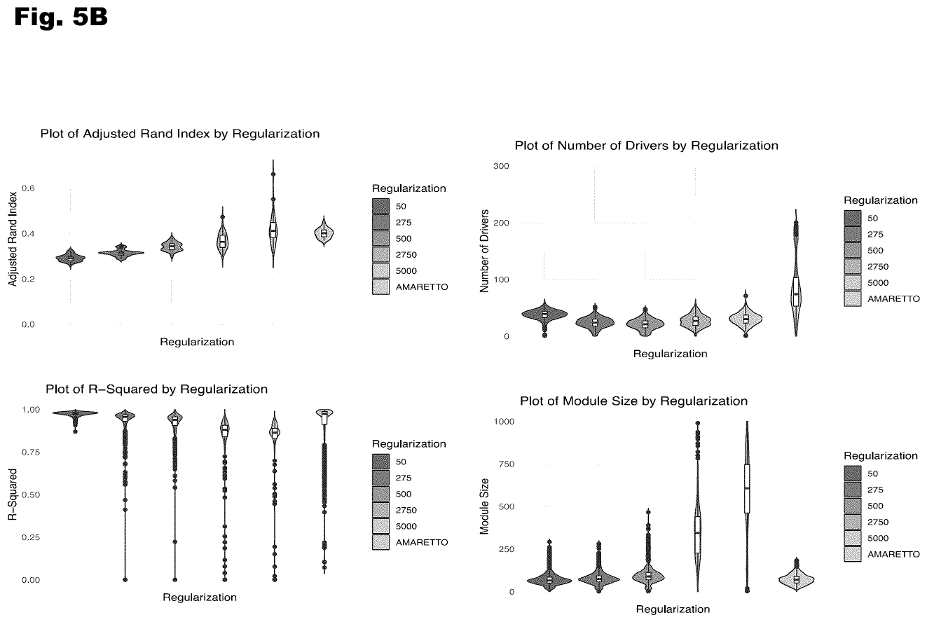

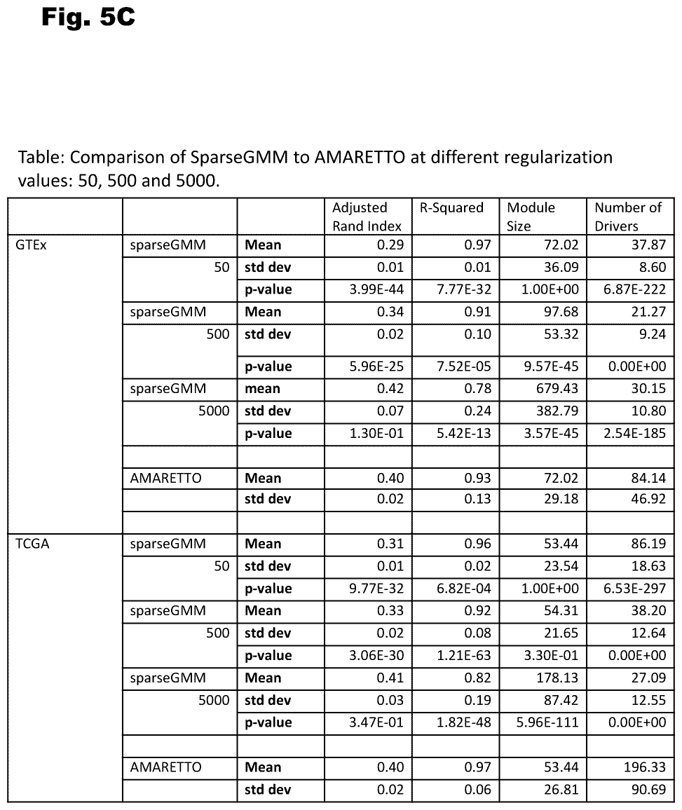

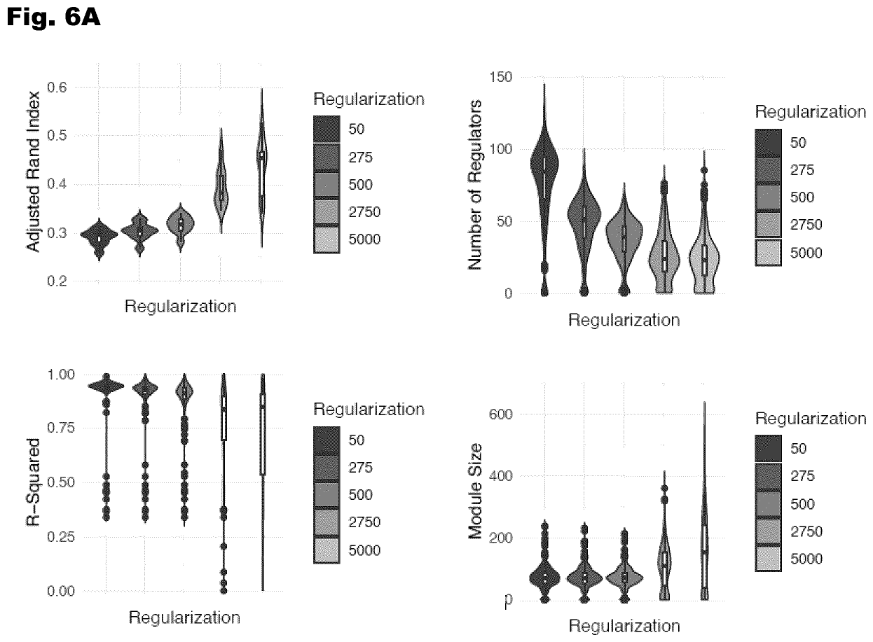

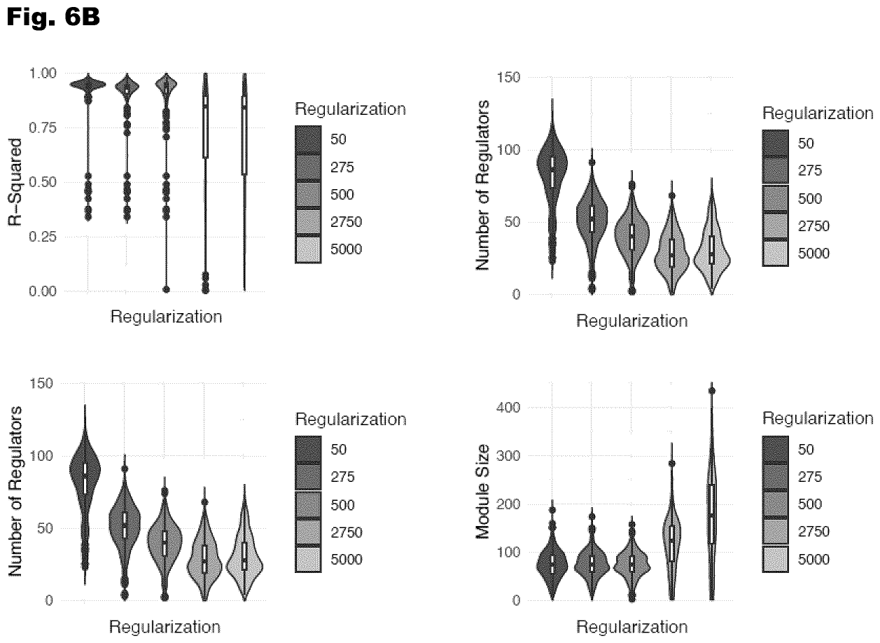

For both TCGA and GTEx data, SparseGMM was compared to AMARETTO (and showed improved performance in terms of sparsity of regulators for various choices of the regularization parameter values (FIGS. 5A to 5C; for more AMARETTO, see O. Gevaert, et al., Interface Focus 3, 20130013, (2013); the disclosure of which is herein incorporated by reference). AMARETTO was selected as a current state-of-the-art method for module-based GRN inference. Analyzing sparsity performance in GTEx and TCGA data, SparseGMM outperforms AMARETTO for all choices of the regularization parameter, lambda, with sparser solutions being more desirable. The mean number of regulators per module, with sparsity parameter lambda=500, is 21.27 for SparseGMM, comparted to 84.14 for AMARETTO using GTEx data (p-value<0.05, independent t-test). Similarly using TCGA data, the mean number of regulators is 38.20 for SparseGMM comparted to 196.33 for AMARETTO (p-value<0.05, independent t-test). For robustness measured using the Adjusted Rand Index (ARI) on modules from multiple runs on each data set, both data sets show similar performance with an increasing trend as the regularization parameter increases. On the other hand, both methods show gradual decrease in R² with increased regularization for both data sets._SparseGMM performs better than AMARETTO for lower values of lambda. Module size increases with regularization for both data sets. At lambda=5e3, SparseGMM has larger module sizes than AMARETTO. At values of lambda>500, the module sizes are too large for practical functional annotation and discovery. Overall, the sparsity performance of SparseGMM was superior for all tested values of the regularization parameter. SparseGMM performed consistently when applied to two other data sets from TCGA: Lung Adenocarcinoma, LUAD and Head and Neck Squamous Cell Carcinomas, HNSC (FIGS. 6A and 6B) Acceptable module sizes and R-squared were seen for lambda=500 and lower similarly, while acceptable adjusted Rand index values were seen at 500 and higher. These results dictated the choice of lambda-500 in subsequent analyses.

### Liver Cancer and Healthy Livers Share an Angiogenesis Community

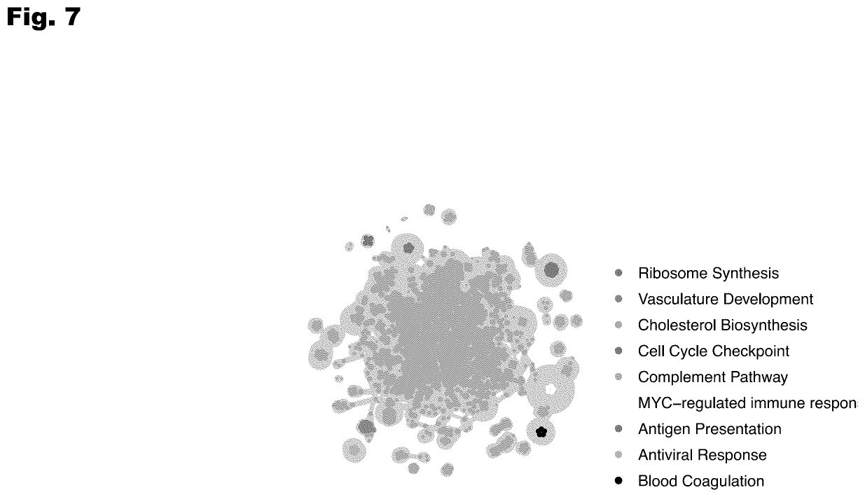

From the combined analysis of normal liver and liver cancer tissue, 72 communities were discovered containing normal liver modules, cancer modules, and communities that combined normal and cancer modules (FIG. 7). Robust communities were defined to be those with an average pairwise Jaccard Index>=0.7 between each two modules. 22/72 such communities were found and the biological function of 15/22 robust communities were reliably identified (See Methods). The Library of Integrated Network-Based Cellular Signatures (LINCS) database was used to validate the uncovered regulatory relationships.

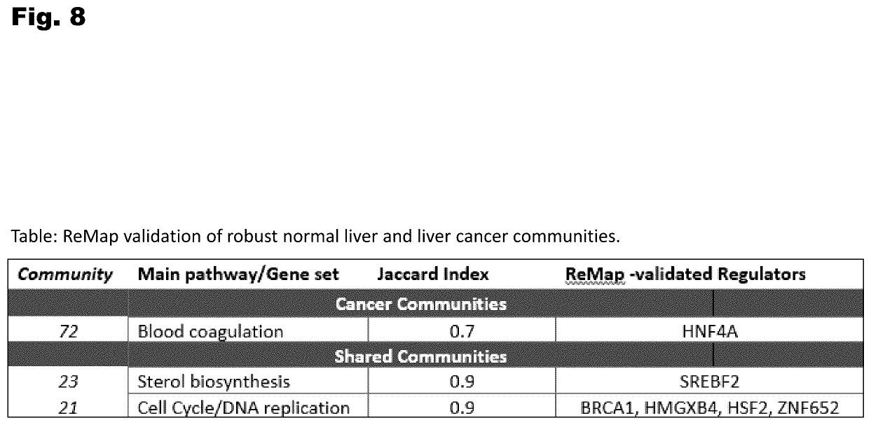

Although many of the regulators do not have corresponding LINCS perturbation experiments, 9 communities (out of 11 non-immune highly robust communities) had at least one regulator validated using LINCS perturbation experiments. For immune communities, known regulators were identified using evidence from previous studies (Supplementary Note 1). ReMap, a database of transcriptional regulators peaks derived from DNA-binding sequencing experiments, was also used to validate robust community regulators. The ReMap database contained data for 10 regulators from six robust communities. The ReMap results showed that six out of ten regulators were able to be validated with ReMap data, including HNF4A (FIG. 8). It was hypothesized that SparseGMM could be useful to find communities shared by both HCC and healthy tissues, leading to the identification of highly conserved functions in HCC. The shared GTEx and TOGA modules were further investigated, revealing four robust communities enriched in functions important for physiological liver regeneration upon damage and tumor growth, including angiogenesis, cell cycle/DNA replication, ribosome, and sterol biosynthesis.

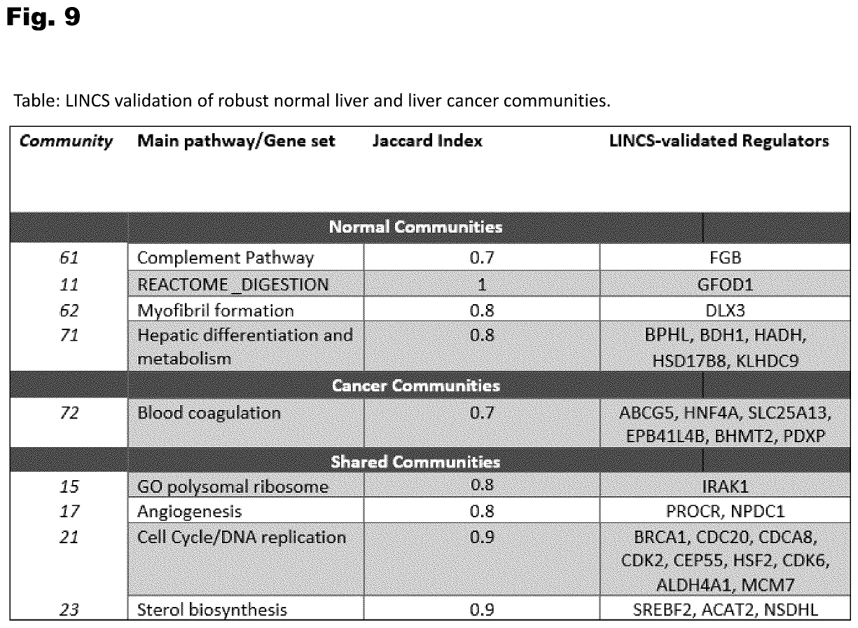

Highlighted here is a shared angiogenesis community that is enriched in gene sets that relate to vasculature development, extension of new blood vessels from existing capillaries into vascular tissues and movement of an endothelial cell to form an endothelium. LINCS perturbation data confirms 2/2 regulators (FIG. 9). The first is NPDC1, a neural factor, which down-regulates cell proliferation. Secondly, PROCR is a receptor of activated protein C, which has a documented role in inhibiting metastasis and limiting cancer cell extravasation through S1PR1. Interestingly, S1PR1 is also a regulator in this community with well-documented roles in angiogenesis and liver fibrosis, but was not validated due to lack of perturbation experimental data in the LINCS database. PROCR was also shown to induce endothelial cell proliferation and angiogenesis and identified as a biomarker of blood vascular endothelial stem cells and a potential cancer biomarker. Among the shared regulators between cancer and normal samples is LDB2, a transcription factor, which regulates the expression of DLL4, a notch ligand Involved in angiogenesis; DLL4 negatively regulates endothelial cell proliferation and migration and angiogenic sprouting.

### Antigen Presentation and Blood Coagulation are Robust Communities Revealed by SparseGMM in HCC

After applying SparseGMM only to HCC gene expression data, five robust communities were discovered that are enriched in pathways with important roles in the interaction between hepatocytes and the immune system: antigen presentation, interferon signaling, myeloid and CD4 and CD8 T cells, and blood coagulation (FIG. 7).

The antigen presentation community included 34 target genes that are directly involved in the process of antigen processing and presentation by the HLA complex to the TCR present in the surface of immune cells. This community is regulated by the PDCD1LG2 gene, encoding PD-L2, an immune checkpoint receptor of PD-1 and a recently adopted revolutionary immunotherapy drug target in HCC patients. The analyses suggest that PDCD1LG2 is a regulator of the myeloid community, while PDCD1 is a regulator the T cell community (Supplementary Note 1).

Next, the community enriched in pathways related to components of the blood coagulation system, and the clotting cascade was examined. This community is also enriched in processes involved in the maintenance of an internal steady state of lipid and sterol, which interact with the coagulation system. Of the 31 regulators in this community, LINCS experimental data was available for 13 genes and 6 (46%) genes were validated (FIG. 9). Among these, HNF4A is the main transcriptional regulator in hepatocytes and regulates multiple coagulation genes. Other validated regulators of this community include EPB41L4B, which promotes cellular adhesion, migration and motility in vitro and is reported to play a role in wound healing. SparseGMM also correctly identified SERPINC1 as a regulator of this community. While there are no LINCS perturbation experiments for SERPINC1, the regulatory role of this member of the serpin family in blood coagulation cascade has been well-documented. These results show that the clotting system is robustly regulated in HCC. While the impact of impaired liver function on blood coagulation is evident, the specific role of this pathway in HCC progression is largely unexplored.

### Sparsegmm Identifies Potential Modules of Hepatic Differentiation and Metabolism in Healthy Livers

Six communities were found that highlight important normal liver functions. GSEA results reveal six distinct functions: Hepatic differentiation and metabolism, lipid, and protein catabolismcomplement, cancer and vesicle trafficking, myofibril formation, and FGFR1 signaling. As an example, the hepatic differentiation and metabolism community was examined further, an important pathway capturing the liver's unique metabolic functions. Specifically, LINCS perturbation experiments validated 50% of regulators (5 out of 10 with available LINCS data) in this community (FIG. 9). Confirmed regulators in this community include two enzymes: BDH1 a short-chain dehydrogenase that catalyzes the interconversion of ketone bodies produced during fatty acid catabolism and HADH responsible for the oxidation of straight-chain 3-hydroxyacyl-CoAs as part of the beta-oxidation pathway. Five target genes in this community were reported as part of a transcriptomic signature of obesity-related steatosis in rat hepatocytes, with functions related to mitochondrial and peroxisomal oxidation of fatty acids, and detoxification. Additionally, it was shown that Bdh1-mediated β-hydroxybutyrylation potentiates propagation of hepatocellular carcinoma stem cells, and that deletion of Bdh1 causes low ketone body level and fatty liver during fasting. Moreover, Bdh1 overexpression ameliorates hepatic injury in a MAFLD mouse model. These findings point to SparseGMM-identified hepatic differentiation and metabolism genes as potential bona fide transcriptional biomarkers of hepatic differentiation and metabolism in healthy livers.

### SparseGMM Decouples Distinct Myeloid and Lymphoid Biological Processes in HCC Micro-Environment, Blood, and Normal Liver

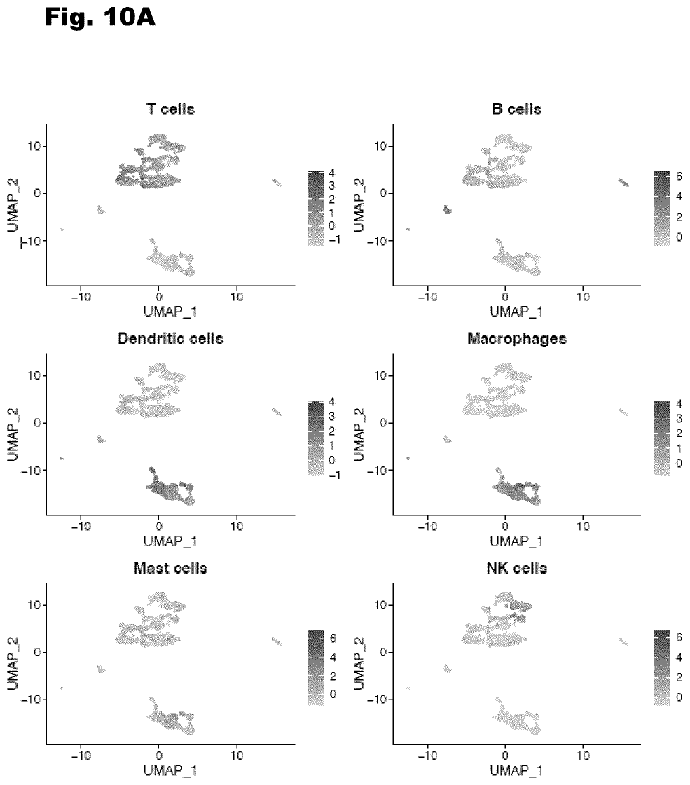

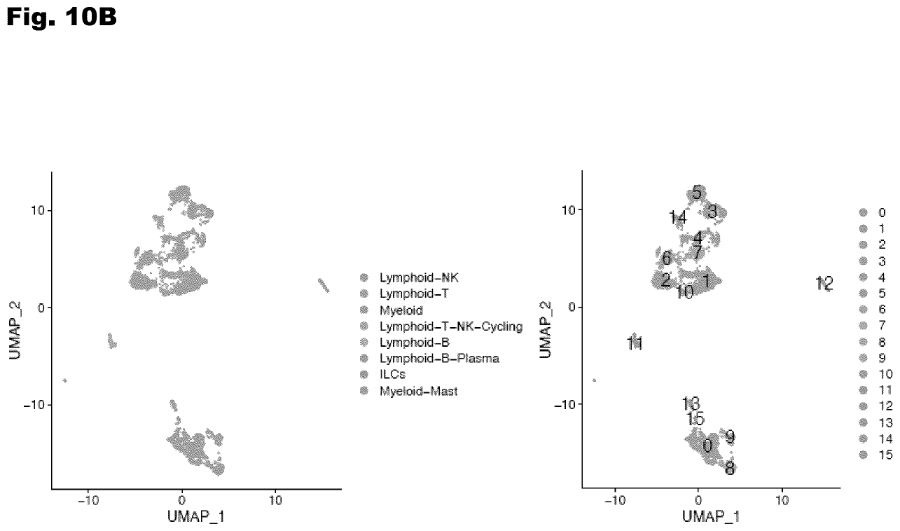

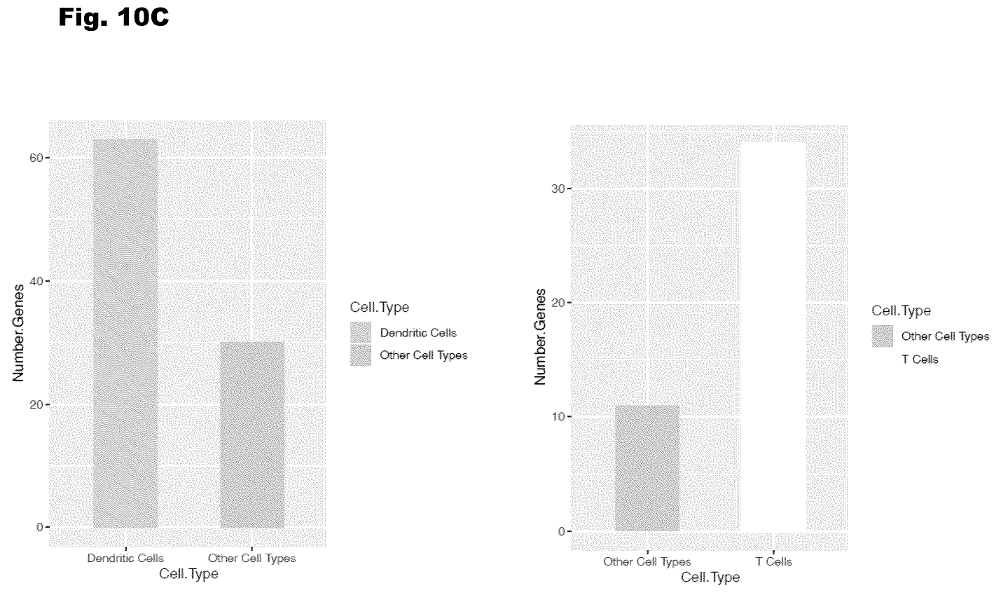

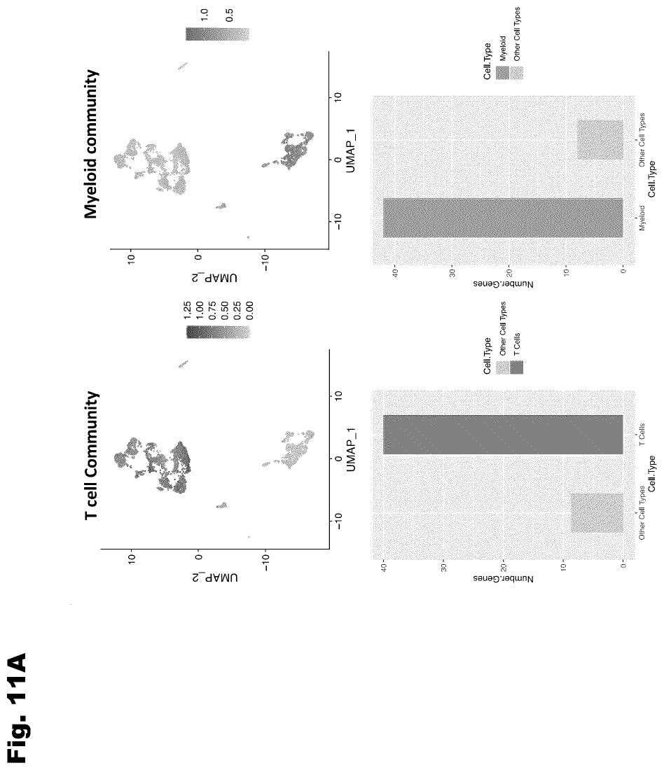

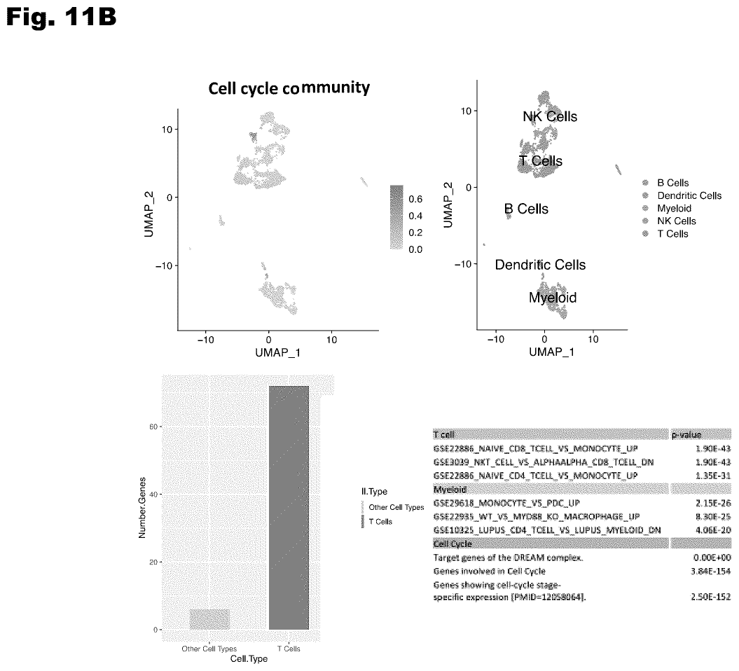

Highly robust communities were examined that were in an independent singe cell RNA data set of CD45+ immune cells for HCC patients from five immune-relevant sites: tumor, adjacent liver, hepatic lymph node (LN), blood, and ascites. Seurat was used to cluster the cells and to compare markers of clusters to markers of various immune cells to identify the different cell types in the tumor samples of three patients (FIGS. 10A to 10C, see Methods). Overall, it was found that 4 out of 9 communities expressed in the single cell data set were cell type specific (FIGS. 10A to 10C, FIGS. 11A and 11B). These communities were CD4 and CD8 T cells community, myeloid community, cell cycle community (specific to T cells and dendritic cells) and the community 60 (specific to T cells).

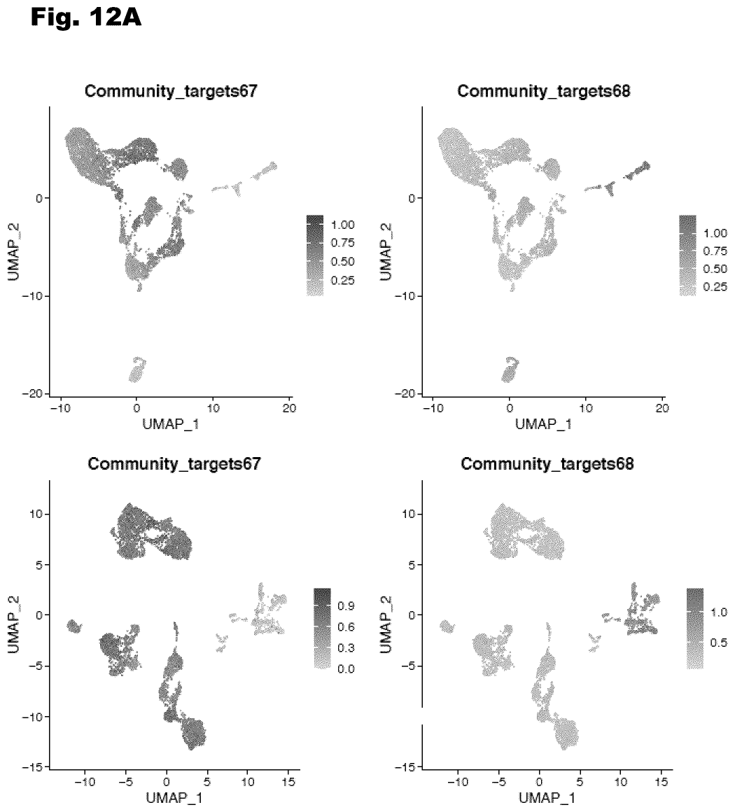

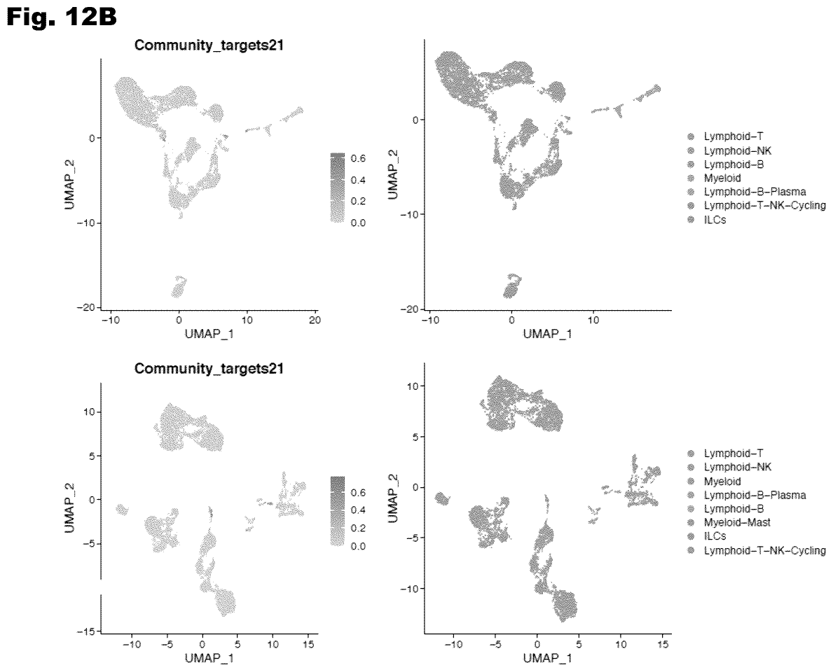

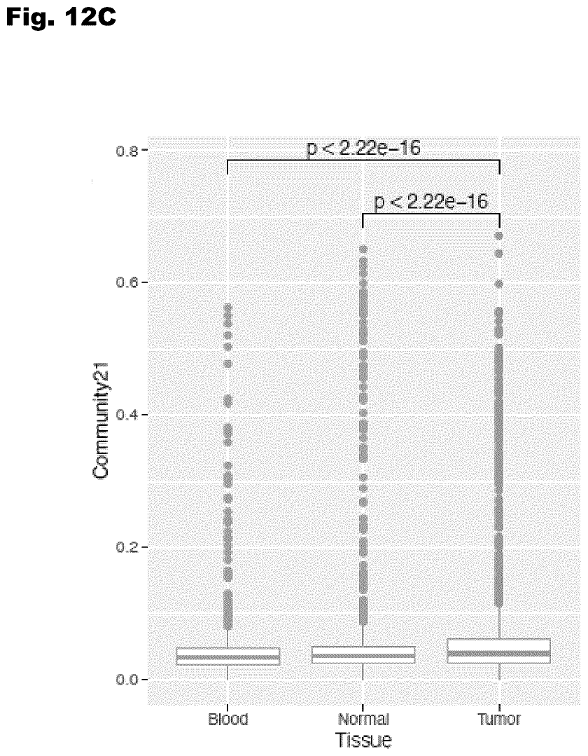

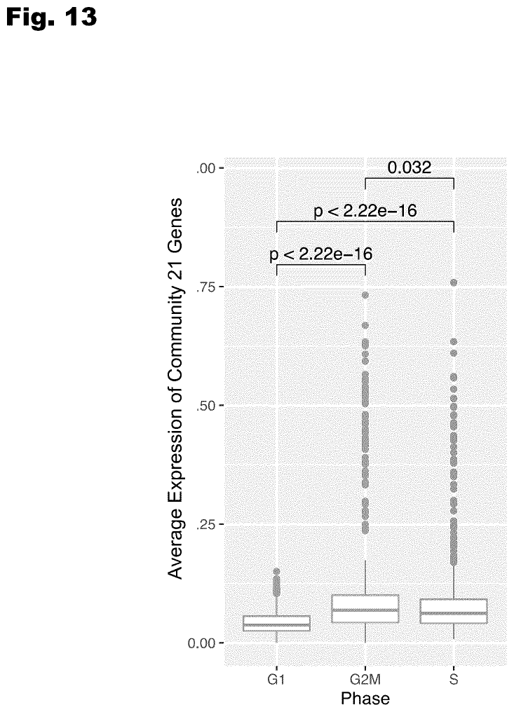

The expression of target genes from communities 67 and 68 distinguished CD4 and CD8 T cells from myeloid cells respectively (FIGS. 11A and 11B) with a similar expression pattern in immune cells from blood and normal liver tissue (FIGS. 12A to 12C). CD4 and CD8 T cells (myeloid cells) expressed a significantly larger number of genes from the CD4 and CD8 T cells community (myeloid cell community) than other cell types (adjusted p-value<0.05, chi-squared test), confirming that the communities are cell-type specific. Additionally, a subset of T cells that specifically express genes from the cell cycle community was observed (adjusted p-value<0.05 chi-squared test). As expected, the cell cycle community gene expression was lower (p-value<2.22e-16, independent t-test) in G1 phase than in the proliferating G2M and S phases (FIG. 13). When comparing this community's average expression in cells from different environments, it was found that the level of cell cycle gene expression is higher in tumor-derived immune cells than in either normal or blood (p-value<2.22e-16, independent t-test, FIGS. 12A to 12C).

Finally, the percentage of variance explained in average target gene expression by regulator expression (R²) was 0.53, 0.80, 0.80 in CD4 and CD8 T cells, myeloid, and cell cycle communities respectively, demonstrating the accuracy of the inferred regulatory programs. These results further support the robustness of communities identified in bulk RNA-sequencing data.

### Gene Entropy Identifies Key Elements of Cancer Pathway Crosstalk

Both in liver physiology and liver cancer, functional crosstalk, defined as the interaction between two pathways belonging to different cell processes, are a natural way of responding to new environmental challenges. Previous studies reported crosstalk between major cancer pathways such as p53 and NF-κB/TNF-α, and p53 and estrogen. Furthermore, this crosstalk between pathways represents compensation mechanisms by which a cancer cell can generate resistance to the blockage of a specific gene or pathway. It was hypothesized that gene entropy (Supplementary Note 2), which is a measure of uncertainty in its assignment to a gene module, could be interpreted as a proxy for multiple module membership, and thus be used to unveil the elements of hidden crosstalk in cancer.

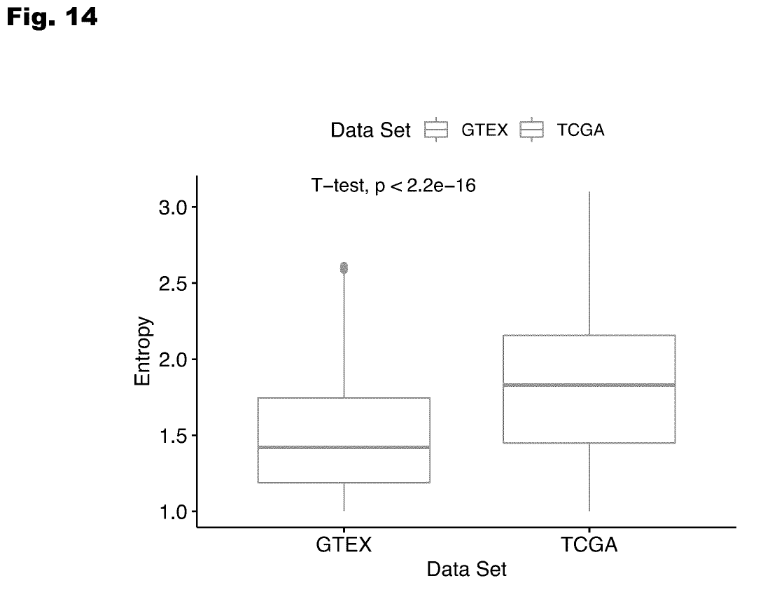

The average entropy of each target gene was calculated over multiple runs of SparseGMM on TCGA and GTEx samples from the genes' posterior probability (see Methods). An entropy threshold of 1 was set, which corresponds to the maximum possible value of entropy between two modules, to identify genes with uncertainty in module assignment. It was found that for target genes with entropy>1, TCGA target genes showed significantly higher degree of entropy when compared to GTEx (p-value<2.22e-16, independent t-test, FIG. 14). This difference in entropy distribution reflects the heterogeneity of cancer tissue compared to normal healthy tissue.

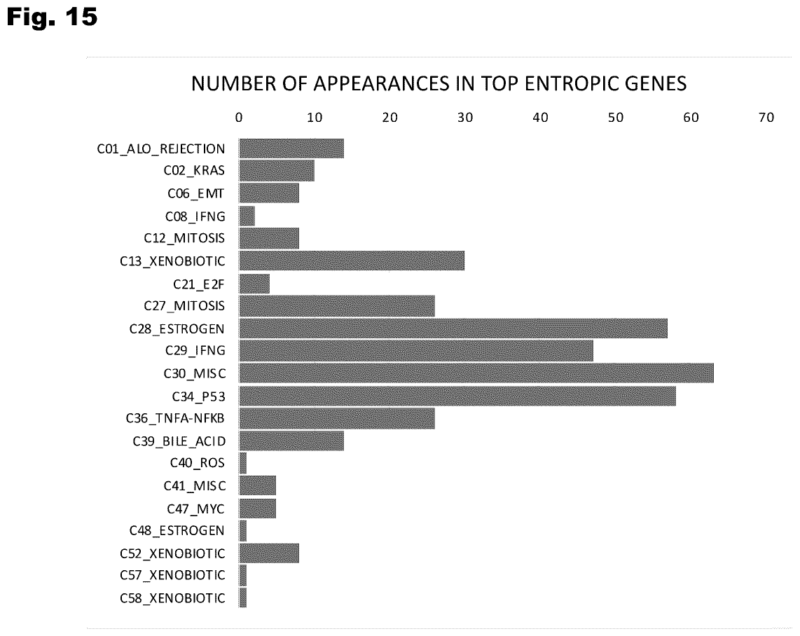

The distribution of community membership was analyzed among high entropy genes in TCGA. Interestingly, genes with high entropy clustered in a few communities such as p53-related networks, NF-κB/TNF-α response, response to interferon-γ, estrogen response, and bile acid metabolism (FIG. 15). p53 harbors loss of function mutation in around one third of patients with HCC. Most HCC originate in an inflammatory liver background such as Hepatitis C or B chronic infection or Nonalcoholic steatohepatitis (NASH), and bile acid composition has been related to HCC. Finally, estrogen signaling has been studied in liver cancer as a potential protective factor and one of the reasons HCC is more frequently seen in males than in females. Altogether, these results suggest an unbiased efficient capturing of clinically relevant pathway crosstalk by SparseGMM. If multifunctional, the genes captured by our method in each crosstalk could be important for identifying key targets for an efficient therapeutic disruption of cancer growth.

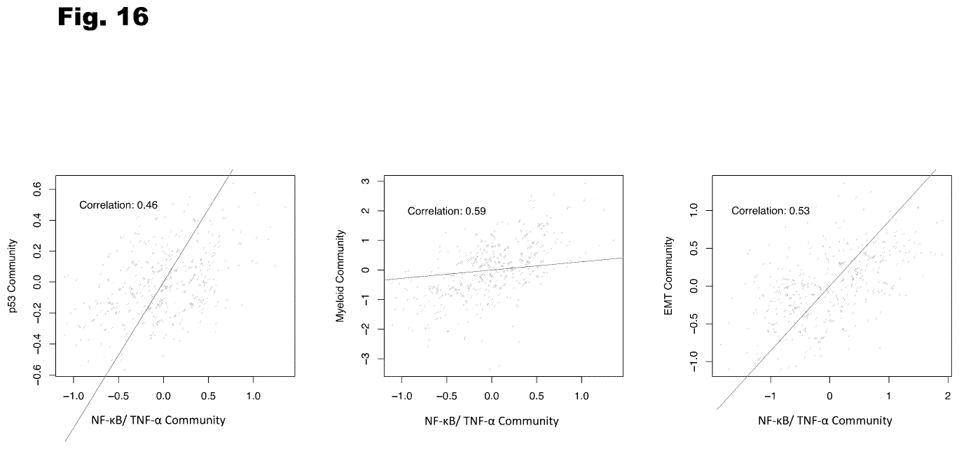

Next, the detected highly entropic genes within crosstalk were further studied. 15 high entropy genes were found that were assigned to both estrogen-mediated signaling and p53 communities. One of these genes, GREB1 is an estrogen-regulated gene that is expressed in estrogen receptor α (ERα)-positive breast cancer cells modulating its function and promoting cancer cell proliferation. The expression of GREB1 is controlled by a p53 target. Similarly, IGFALS is another high entropy gene with assignment to both p53 and estrogen signaling communities. This is consistent with the fact that IGFALS is interacts with a p53 target, and has a role in regulating estrogen receptor in breast cancer. Additionally, the crosstalk between p53 and NF-κB/TNF-α pathways was examined more closely. PAX8, a transcription factor expressed in 90% of high degree serous carcinoma, is among the highly entropic genes identified by SparseGMM as participating in both p53 and TNFα/NFkB1-related signaling. Interestingly, a significant correlation between average target gene expression of p53 and NF-κB/TNF-α pathways was found (FIG. 16, left panel, Pearson correlation=0.46 Cl [0.37-0.53], p-value <2.2e-16). Previous studies also showed that p53 and NF-κB/TNF-α coregulate proinflammatory gene responses in human macrophages. significant correlation between the TNF-α induced NF-κB community and the myeloid community was observed (FIG. 16, middle panel, Pearson correlation=0.48, Cl [0.41-0.56], p-value <2.2e-16). The p53-NF-κB/TNF-α crosstalk is also implicated in increased invasiveness. A significant correlation between the NF-κB/TNF-α community and the Epithelial to Mesenchymal Transition (EMT) and cancer stemness community was found (FIG. 16, right panel, Pearson correlation=0.53, Cl [0.45-0.60], p-value <2.2e-16). Accordingly, SparseGMM is able not only to infer key regulators and their downstream gene modules, but also potentially identify key multifunctional components shared by critical cancer pathways based on their entropy.

### Materials and Methods

### Data Preprocessing

Gene modules in normal tissue were constructed using publicly available data from GTEx project, while cancer modules were constructed using HCC data from TCGA project. The data sets were preprocessed, in which reference RNA-seq expression levels were provided that were from healthy human tissue that can be compared with the expression levels found in human cancer tissue.

A list of candidate genes was obtained from previously generated AMARETTO data objects extracted using the TFutils R package. In addition, genes whose gene expression can be explained using changes in copy number variation or methylation status from the TCGA data set were extracted using the AMARETTO package. The combined list of genes was used as an initial candidate regulator gene list. Next, the top 75% varying genes were identified to each data set separately. Of the top 75%, the top 2000 genes that are also present in the candidate regulator genes list were used to build the regulator gene matrix, the rest were regarded as target genes. The gene expression data matrix was centered to mean 0 and standard deviation 1 and then split into a regulator gene matrix and a target gene matrix. A similar approach was used to preprocess and build the input data matrices from the GTEx data set. Overall, the TCGA contained 8017 protein-coding genes including 2000 candidate drivers, while the GTEx network contains 10804 protein-coding genes including 1800 candidate drivers.

### Implementation and Technical Validation

SparseGMM (Supplementary Note 2) was implemented in Python. SparseGMM was run 5 times on each data set with different seeds to evaluate the robustness of the method. both data sets were split into training (70%) and test (30%) sets. Four different metrics were employed to validate the performance of SparseGMM: 1) Adjusted index (ARI) to measure robustness, 2) R-Squared to measure goodness of fit, 3) Number of selected regulators to measure sparsity, and 4) Size of module to evaluate the sensitivity of module size to the regularization parameter lambda. The values of lambda above 5000 produced very large modules and were excluded from further analysis. Values below 50 were also excluded due to low adjusted rand index. Lambda values examined were 50, 275, 500, 2750 and 5000 were used. The output of different seeds was also used to filter the generated modules using cAMARETTO as explained below. The input number of clusters used was 150 as it resulted an average size of 60 genes per cluster and reduced false positive results in downstream functional gene set enrichments (FIGS. 5A and 5B).

### Robust Module Recovery Via Community Detection

To detect robust modules, the community-AMARETTO (cAMARETTO) package (O. Gevaert, et al., JCO Clin Cancer Inform 4, 421-435, (2020); the disclosure of which is herein incorporated by reference) was used to build communities among modules discovered by running SparseGMM with different seeds on the same data set. cAMARETTO identifies gene modules and their regulators that are shared and distinct across multiple regulatory networks. Specifically, cAMARETTO takes as input multiple networks inferred using the SparseGMM algorithm. cAMARETTO can learn communities or subnetworks from regulatory networks derived from multiple cohorts, diseases, or biological systems. To do this cAMARETTO uses the Girvan-Newman “edge betweenness community detection” algorithm. The cAMARETTO algorithm consists of 1) constructing a master network composed of multiple regulatory networks followed by 2) detecting groups (communities) of modules that are shared across systems, as well as highlighting modules that are system-specific and distinct. By applying cAMARETTO to modules discovered by running SparseGMM with different seeds on the same data set, modules that are consistently discovered by SparseGMM will be grouped in the same subnetwork or community, i.e., copies of the same module will be clustered in a distinct community. cAMARETTO parameters used were p-value=0.01 and intersection=10. When running cAMARETTO on a single data set (either GTEx or TCGA), filtering for communities of size 5 was done, one from each run and further narrowed down results by Jaccard index>=0.7. In contrast, for communities with both TOGA and GTEx modules, communities of size 10 were selected. The selected communities were used as input to the GSEA function of cAMARETTO.

### Gene Set Enrichment Analysis

GSEA was applied using MSigDB collections (Hallmarks and C1-5) to functionally annotate each of the communities. A p-value<1e-5, adjusted for testing of multiple hypotheses using the Benjamini-Hochberg method, was selected to filter enriched data sets.

### Biological Validation

To experimentally validate regulators of the discovered communities, the robust regulators were interrogated, which were defined as regulators consistently associated with the same community by SparseGMM across all runs, against publicly available genetic perturbation studies in the Library of Integrated Network-Based Cellular Signatures (LINCS) database. In this validation experiment the HEPG2 liver cell line data was leveraged. The types of perturbation experiments used were 1) Consensus signature from shRNAs targeting the same gene and 2) cDNA for overexpression of wild-type gene. The Fast Gene Set Enrichment Analysis tool was used to test for significance in enrichment. To empirically derive p-values, 1000 lists of genes of the same size were permuted as the community target sets for each community and for each regulator. Regulator-gene set pairs which had a corresponding p-value<0.05, adjusted for testing of multiple hypotheses using the Benjamini-Hochberg method, were considered validated cellular signatures in either of the two signature types.

ReMap (F. Hammal, et al., Nucleic Acids Res 50, D316-D325, (2022); the disclosure of which is herein incorporated by reference) was also used, a database of transcriptional regulators peaks derived curated from DNA-binding sequencing experiments to validate our robust community regulators. Analysis was restricted to experiments on the HEPG2 liver cell line data. A hypergeometric test was used to test for significance between ReMap data and the generated data, the Bonferroni method was used to correct for multiple comparisons.

### Single Cell Transcriptomic Evaluation

The highly robust communities were evaluated in an independent singe cell RNA data set with samples from immune-relevant sites in five HCC patients: tumor, adjacent liver, hepatic lymph node (LN), blood, and ascites (Accession number: GSE140228, Gene Expression Omnibus). This data set contains only purified CD45+ immune cells and no other cell types. The expression in tumor core was used to evaluate the generated communities, which was available from three patients. Preprocessing procedure was as follows: single cells were processed through the GemCode Single Cell Platform using the GemCode Gel Bead, Chip and Library Kits (10× Genomics, Pleasanton). The cells were partitioned into Gel Beads in Emulsion in the GemCode instrument, where cell lysis and barcoded reverse transcription of RNA occurred, followed by amplification, shearing and 30 adaptor and sample index attachment. Libraries were sequenced on an Illumina Hiseq 4000. Seurat was used to analyze the data set (T. Stuart, et al., Cell 177, 1888-1902 e1821 (2019); the disclosure of which is herein incorporated by reference). To perform quality control of the data, genes that were expressed in less than 40 cells were filtered and cells that had fewer than 1000 or greater than 5000 genes were filtered, and cells with a proportion of transcripts mapping to mitochondrial genes greater than 5% were filtered. Data was scaled before applying PCA, then clustering and UMAP using the top 10 PCA dimensions. The resulting Seurat clusters and PanglaoDB cell markers were used to identify cell marker expression. The average expression of cell type markers was compared to annotate cells and compared Seurat clusters to PanglaoDB annotations to assign an immune cell type to each cluster in the core tumor samples. To evaluate the expression of the generated communities, and the cell-type specificity of each community, the number of genes expressed from each community in each cell type were compared to the average number of genes expressed in other cell types using a chi-squared test. Seurat was used to score the cell cycle phase of cells.

### Supplementary Note 1: Identifying Key Multifunctional Components Shared by Critical Cancer and Normal Liver Pathways Via Sparsegmm

### Normal Liver Communities

A community was discovered, which is enriched in gene sets related to the complement pathway (FIG. 5C). The complement system has as a critical role in immune response and liver hepatocytes are responsible for production of complement proteins. FGB, the gene encoding the beta component of fibrinogen was confirmed by LINCS perturbation data as a regulator. Fibrinogen is a precursor of fibrin, the most abundant component of blood clots. There are many links between coagulation and innate immunity, including the role of fibrin in activating the complement system. However, this finding suggests a regulatory role of fibrin precursor in the production of complement proteins. SparseGMM also discovered CFH, a known regulator of the complement system, with no LINCS perturbation data available. CFH is a member of the regulator of complement activation gene cluster, which plays an essential role in the regulation of complement activation.

Another highly robust community is enriched in gene sets related to digestion pathways (FIG. 5C). The community contains modules of the several digestive enzyme groups: lipases, liver amylase and proteases. robust drivers that regulate all of the modules present in this community were examined and data from the LINCS perturbation library was used to confirm these regulatory relationships. LINCS analysis confirmed GFOD1 gene, a glucose-fructose oxidoreductase, as a regulator of the community.

### Hepatocellular Carcinoma Communities

A community enriched for T cell upregulated genes was discovered, showing a strong co-expression pattern of these genes in both bulk and single cell data. Specifically, this community is regulated by the immune checkpoint gene: PDCD1, which encodes for the PD-1 protein. The community also contains the immune checkpoint gene CTLA4, as well markers of memory CD8 resident T-Cells: CD8, CD2, CD48. CXCR3, which plays a role in T cell trafficking and function, and ZNF683 a known tissue-resident lymphocyte transcription factor are also members of this community. Finally, this community includes T-cell receptor complex genes: CD3 epsilon, gamma, delta, and CD247.

Next, is a community enriched for myeloid up-regulated genes. This community is regulated by PDCD1LG2, which encodes PD-L2, an immune checkpoint receptor ligand of PD-1. This community is also regulated by CD74, a cell surface molecule with a critical role in antigen presentation. The community also contains several members of the MHC class II genes.

### Shared Communities

First, community 21 is enriched in cell cycle gene sets. In this community, 10/30 genes were validated using LINCs perturbation data. In this shared community, cancer and normal modules shared 2 out of the 10 confirmed regulators: CDC20, a regulatory protein interacting with several other proteins at multiple points in the cell cycle and CDCA8 component of the chromosomal passenger complex, which is a regulator of mitosis and cell division. The remaining 8 regulators were specific to cancer modules. It was expected that most of the validated regulators would be either shared or cancer-specific, since LINCS perturbation experiments are carried out using cancer cell line data. A similar regulatory pattern was expected for the regulators discovered specifically in normal samples.

Another shared community is enriched for gene sets related to protein synthesis and formation of ribosomal subunits, ubiquitin ligase inhibitor activity and granular component. LINCS perturbation data is available for four regulators. LINCS data analysis confirms one cancer-specific regulator, IRAK1, which does not regulate this community in normal tissue. IRAK1 plays a critical role in initiating innate immune response against foreign pathogens. IRAK1 was also shown to promote cell proliferation and protect against apoptosis in HCC and to regulate the properties of liver tumor-initiating cells (TIC), including self-renewal, and tumorigenicity, suggesting IRAK1 as a potential therapeutic target of HCC.

Finally, LINCS perturbation results confirmed 50% of regulators with available experimental data (3 out of 6) in the sterol biosynthesis community including ACAT2 and SREBF2. ACAT2, an enzyme involved in lipid metabolism and cholesterol esterification, was associated with tumor growth and progression in liver and colorectal cancers. Additionally, the blockage of SREBF2, a master transcriptional regulator of cholesterol and fatty acid pathway genes was shown to completely prevent hepatocarcinogenesis and impaired the survival of HCC cell lines through the suppression of AKT-mTORC-RPS6 dependent cell proliferation. This community contains 20 target genes, all of which are associated with cholesterol and lipid metabolism. Thus, this community reflects both an essential metabolic liver function, as well as an accelerated sterol and lipid metabolism that is a hallmark of cancer. These findings point towards an importance of sterol biosynthetic expression programs for tumor growth.

### Supplementary Note 2; Model for Sparse Gaussian Mixtures

### Model

A Bayesian generative model was constructed for learning the regulatory relationships among genes. In the context of gene regulatory networks, genes were classified into one of two types: target genes and regulator genes. Regulator genes are genes undergoing genomic events that are relevant to cancer progression or tumor growth. Target genes are genes whose expression is controlled by regulator genes, and which contribute to the biological processes responsible for cancer progression. Each group of target genes is regulated by a small set of regulator genes.

This model can be formulated as follows: X^{T}=[x₁x₂ . . . xᵢ . . . x_{N}] is a gene expression matrix X ∈ [symbol] ^{N×M} where Nis the number of target genes and Mis the number of subjects, G is a regulator expression matrix E RMxP where M is the number of subjects and P is the number of regulator genes. Finally, β ∈ [symbol] ^{P×K} is a weight matrix, where K is the number of gene modules. The mean of each Gaussian component is a vector of weights passed through a constant regulator gene expression matrix:

$$z \vert \sim Cat ( π ) \\ {x}_{i} \vert {z}_{i} = k \sim 𝒩 ( G {β}_{k} , {σ}_{k} I ) ,$$

where zᵢ is the latent indicator of the mixture component that generated gene i. The expression of gene i is a sample from a Gaussian with mean equal to the weights, βₖ passed through a constant regulator gene expression matrix G. σₖ is the variance of the Gaussian mixture component k and π is the parameter of the categorical distribution. Thus, θₖ=[βₖ, σₖ].

This Bayesian approach combines Gaussian mixtures with l1-norm regularization to enforce sparsity on the regulator weights, resulting in a small set of regulators for each mixture component. An expectation-maximization (EM)-based algorithm was developed to obtain a maximum a posteriori (MAP) estimate the Gaussian mixture of parameters. This is detailed in the following sections.

### Gaussian Mixtures for Gene Regulatory Networks

Mixture models are useful for representing data that are generated from different distributions, such as multimodal data. The data is assumed to be generated from a mixture of components, each with specific parameters that specify its distribution. The goal is to estimate these parameters using the observed data without observing the true component membership of the data points, which is a hidden or latent variable of the model. In a mixture model with K distributions zᵢ∈{1, . . . , K}, point xᵢ is generated from distribution k with likelihood p(xᵢ|zᵢ=k). zᵢ has the distribution p(zᵢ)=Cat(π) and the K distributions are mixed as follows:

$$p ( {x}_{i} \vert θ ) = {\sum }_{k = 1}^{K} {π}_{k} p ( {x}_{i} \vert {θ}_{k} ) , \qquad ( 1 )$$

where θ are the parameters to be estimated for k=1: K, θ is [θ1 . . . θₖ . . . θ_{K}]. π_{K} is the mixing weight of base distribution k, 0<πₖ<1 and Σₖ₌₁^{K}πₖ=1. For example, a mixture of Gaussian distributions would be modeled as follows:

$$p ( {x}_{i} \vert θ ) = {\sum }_{k = 1}^{K} {π}_{k} 𝒩 ( {x}_{i} \vert {μ}_{k} , {Σ}_{k} ) , \qquad ( 2 )$$

Point i can then be assigned to a component using the MAP or ML estimate of the parameter θ is needed.

To obtain this estimate, the model was fit for the data [symbol] , using the iterative expectation-maximization (EM) algorithm applied to the likelihood function. The EM algorithm, consists of two steps. In the first (E) step the missing values are inferred using parameter estimates from the previous iteration. In the second (M) step, the likelihood function is maximized with respect to model parameters, giving new parameter estimates, which are improved with each subsequent iteration until convergence.

Using this model to cluster the data involves calculating the posterior probability p(zᵢ=k|xᵢ,θ^{t-1}), the posterior probability that point i is generated from distribution k or the responsibility of cluster k for point i:

$${r}_{ik} {=}^{△} p ( {z}_{i} = k \vert {x}_{i} , {θ}^{( t - 1 )} ) , \qquad ( 3 )$$

where t is the current iteration number. This can be expanded as:

$${r}_{ik} = \frac{p ( {z}_{i} = k \vert {θ}^{t - 1} ) p ( {x}_{i} \vert {z}_{i} = k , {θ}^{t - 1} )}{{\sum }_{{k}^{′}} p ( {z}_{i} = {k}^{′} \vert {θ}^{t - 1} ) p ( {x}_{i} \vert {z}_{i} = {k}^{′} , {θ}^{t - 1} )} . \qquad ( 4 )$$

To derive the objective function, the complete log likelihood of the data is computed, which is defined as:

$${ℓ}_{c} ( θ ) {=}^{△} {\sum }_{i = 1}^{N} log [ p ( {x}_{i} , {z}_{i} \vert θ ) ] . \qquad ( 5 )$$

Since the cluster assignments, zᵢ are not observed the expected likelihood is used. This is defined as:

$$Q ( θ , {θ}^{t - 1} ) = 𝔼 [ {ℓ}_{c} ( θ ) \vert 𝒟 , {θ}^{t - 1} ] , \qquad ( 6 )$$

where the expectation was taken to account for the fact that zᵢ is not observed.

Specifically in the case of GMM, this gives:

$$Q ( θ , {θ}^{t - 1} ) = {\sum }_{i = 1}^{N} {\sum }_{k = 1}^{K} {r}_{ik} log [ {π}_{ik} p ( {x}_{i} \vert {θ}_{k} ) ] . \qquad ( 7 )$$

Now, a MAP estimate can be performed on the above equation in the M step, obtaining θ^{K}. In the case of Gaussian mixtures, each class conditional density is a Gaussian distribution and θ is made up of the mean and variance of each distribution and this estimate is iteratively improved. Upon convergence, the final iteration T gives the final estimate θ^{T}. This model can be applied to gene regulatory networks. In this case, the average expression of target genes is the mean of the mixture component, which corresponds to a gene module. The mean of the component is a linear function of the regulator genes regulating that module. Equation (7) then becomes:

$$Q ( θ , {θ}^{t - 1} ) = {\sum }_{i = 1}^{N} {\sum }_{k = 1}^{K} {r}_{ik} log [ {π}_{ik} 𝒩 ( {x}_{i} \vert G {β}_{k} , {σ}_{k}^{2} ) ] . \qquad ( 8 )$$

### Map Estimation-l1-Norm Regularization and SparseGMM

MAP estimation with the right prior can be useful to avoid over-fitting of parameter estimates, which can occur in the case of Maximum Likelihood Estimation (MLE). Adding parameter priors, (8) becomes:

$$Q ( θ , {θ}^{t - 1} ) = {\sum }_{i = 1}^{N} {\sum }_{k = 1}^{K} {r}_{ik} log [ {π}_{ik} 𝒩 ( {x}_{i} \vert G {β}_{k} , {σ}_{k}^{2} ) ] + log ( p ( θ ) ) . \qquad ( 9 )$$

The parameters of the GMM to be estimated are θₖ=[βₖ, σₖ] and πₖ for k=1: K. In this problem, there is more interest in discovering the regulatory relationships between regulator and target genes, so a zero-mean Laplace prior was used for the weights βₖ and uniform priors was used for σₖ and πₖ. Uniform priors will give the same result as MLE estimates, while the Laplace prior will give a regularized MAP estimate. Specifically, a Laplace prior is commonly chosen where a sparse solution is desired as it corresponds to l1-norm regularization. A sparse solution can improve understanding of gene regulatory relationships, as it was hypothesized that only a few regulator genes regulate each module.

The expected likelihood function from (9) is updated to be:

$$Q ( θ , {θ}^{t - 1} ) = \qquad ( 10 )$$

$${\sum }_{i = 1}^{N} {\sum }_{k = 1}^{K} {r}_{ik} log [ {π}_{ik} 𝒩 ( {x}_{i} \vert G {β}_{k} , {σ}_{k}^{2} ) ] ++ log [ Lap ( {β}_{k} \vert 0 , 1 / {γ}_{k} ) ] +$$

$$log ( p ( {σ}_{k} ) + log ( p ( {π}_{k} ) ) ,$$

where

$$log ( p ( θ ) ) = {\sum }_{i = 1}^{N} {\sum }_{k = 1}^{K} log [ Lap ( {β}_{k} \vert 0 , 1 / {γ}_{k} ) ] + log ( p ( {σ}_{k} ) + log ( p ( {π}_{k} ) ) . \qquad ( 11 )$$

Thus, a sparse Gaussian Mixture model was defined as a Gaussian mixture model, where the mean of each Gaussian component is a random vector of weights sampled from a Laplace distribution with zero mean and passed through a constant matrix.

### Hierarchical Bayes Modeling

Using a Laplace prior directly results in an l1-norm, which does not give a closed form solution during optimization.
an approach similar to the EM for lasso approach was followed (K. P. Murphy, Machine learning: a probabilistic perspective. MIT press, 2012; the disclosure of which is herein incorporated by reference). The representation of a Laplace distribution was then utilized as a Gaussian Scale Mixture (GSM) (see D. F. Andrews and C. L. Mallows, Journal of the Royal Statistical Society: Series B (Methodological), vol. 36, no. 1, pp. 99-102, 1974; and M. West, Biometrika, vol. 74, no. 3, pp. 646-648, 1987; the disclosures of which are herein incorporated by reference).

$$Lap ( {β}_{p} \vert 0 , 1 / γ ) = \frac{γ}{2} {e}^{- γ \vert {β}_{p} \vert} = \int 𝒩 ( {β}_{p} \vert 0 , {τ}_{p}^{2} ) Ga ( {τ}_{p}^{2} \vert 1 , \frac{{γ}^{2}}{2} ) d {τ}_{p}^{2} . \qquad ( 12 )$$

This is an example of a hierarchical Bayes model, where a prior was included on the hyperparameter τ² of the prior distribution p(θ). In this case, the hyperprior is the Gamma distribution with scale parameter γ²/2. The expected complete data log likelihood is given by:

$$Q ( θ , {θ}^{t - 1} ) = {\sum }_{i = 1}^{N} {\sum }_{k = 1}^{K} {r}_{ik} log [ {π}_{ik} 𝒩 ( {x}_{i} \vert G {β}_{k} , {σ}_{k}^{2} ) ] + \int log [ 𝒩 ( {β}_{k} \vert 0 , {D}_{τ k} ) [ {\sum }_{p} Ga ( {τ}_{kp}^{2} \vert 1 , {γ}^{2} / 2 ) ] ] d {τ}_{kp} + log ( p ( {σ}_{k} ) + log ( p ( {π}_{k} ) ) , \qquad ( 13 )$$

where D_{τk} is diag (τₖₚ²) for p=1: K. The objective function then becomes:

$$Q ( {β}_{k} , {σ}_{k} ) = \qquad ( 14 )$$

$${\sum }_{i = 1}^{N} [ {r}_{ik} [ - n log {σ}_{k} - \frac{1}{2 {σ}_{k}^{2}} {‖ {x}_{i} - \vert G {β}_{k} ‖}_{2}^{2} + log ( {π}_{ik} ) ] - \frac{1}{2} {β}_{k}^{T} {Λ}_{k} {β}_{k} ) d τ ] + c ,$$

where: πᵢₖ is the marginal probability of component zᵢ=k, Λₖ=diag (1/τₖₚ²) for p=1: K and

$$c = {\sum }_{k = 1}^{K} \int [ log ( p ( {τ}_{k p} ) ) ] d τ + log ( p ( {σ}_{k} ) + log p ( π ) ) .$$

### EM Algorithm

E step: Evaluate: E(1/τ²) and rᵢₖ. From the expected complete data likelihood equation (14), the expected value of Λₖ is

$$E [ {Λ}_{k} [ {\hat{β}}_{k} , x , {\hat{σ}}_{k} ] = γ diag ( \vert {\hat{β}}_{1 k}^{{}_{{}_{- 1}}} \vert - \vert {\hat{β}}_{Pk}^{{}_{{}_{- 1}}} \vert ) . \qquad ( 15 )$$

and the responsibilities are:

$${r}_{ik} = \frac{{π}_{k} p ( {x}_{i} , {β}_{k ,} {σ}_{k} )}{{\sum }_{{k}^{\text{?}}} {π}_{k} , p ( {x}_{i} , {β}_{k ,} {σ}_{k} )} \text{?} \qquad ( 16 )$$

$$\text{?} \text{indicates text missing or illegible when filed}$$

where

$$p ( {x}_{i} \vert G , {β}_{k} , {σ}_{k} ) = 𝒩 ( {x}_{i} \vert G {β}_{k} , {σ}_{k} {I}_{N} ) , \qquad ( 17 )$$

and

$$p ( {β}_{k} \vert {τ}_{k} ) = 𝒩 ( {β}_{k} \vert 0 , {D}_{τk} ) . \qquad ( 18 )$$

M step: Using a sparse learning approach (M. A. Figueiredo, IEEE transactions on pattern analysis and machine intelligence, vol. 25, no. 9, pp. 1150-1159, 2003; the disclosure of which is herein incorporated by reference). model parameters πₖ, βₖ and σₖ were estimated by optimizing the expected complete likelihood function with respect to each of the parameters, after substituting E(1/τ²) and rᵢₖ obtained in the E step, taking derivative wrt βₖ

$${∇}_{{β}_{k}} {\text{?}}_{\text{?}} = {\sum }_{i = 1}^{N} {r}_{ik} [ {G}^{T} {x}_{i} - {G}^{T} G {β}_{k} ] - {σ}_{k} {Λ}_{k} {β}_{k} ] = 0 \\ ( {\sum }_{i = 1}^{N} {r}_{ik} {G}^{T} G + {σ}_{k} {Λ}_{k} ) {β}_{k} = {\sum }_{i = 1}^{N} {r}_{ik} ( {G}^{T} {x}_{i} ) \\ [ ( {r}_{k}^{T} 1 ) {G}^{T} G + {σ}_{k} {Λ}_{k} ] {β}_{k} = ( {G}^{T} {X}^{T} ) {r}_{k}$$

$$\text{?} \text{indicates text missing or illegible when filed}$$

where rₖ is the responsibility vector of component k ∈ [symbol] ^{N}

$${\hat{β}}_{k} = {( ( {r}_{k}^{T} 1 ) {G}^{T} G + {σ}_{k} {Λ}_{k} )}^{- 1} ( {G}^{T} {X}^{T} {r}_{k} ) . \qquad ( 19 )$$

Taking the derivative wrt σₖ yields

$${\hat{σ}}_{k} = \frac{{\sum }_{i = 1}^{N} {r}_{ik} {‖ xi - G {\hat{β}}_{k} ‖}_{2}^{2}}{M ( {r}_{k}^{T} 1 )} . \qquad ( 20 )$$

Taking the derivative wrt πₖ yields the same result as a GMM:

$${\hat{π}}_{k} = \frac{{r}_{k}^{T} 1}{N} . \qquad ( 21 )$$

### Implementation for Numerical Stability

Since most βₖ was expected to be equal to zero, and to make the matrix under the inverse numerically stable, the SVD decomposition of G was use as follows:

$$G = U {DV}^{T} , \qquad ( 22 )$$

and

$$ψ = diag ( \vert {β}_{jk} \vert / \text{?} ) . \qquad ( 23 )$$

$$\text{?} \text{indicates text missing or illegible when filed}$$

Taking the derivative wrt βₖ:

$${argmax}_{{β}_{k}} {\sum }_{i = 1}^{N} {r}_{ik} [ \frac{1}{{σ}_{k}} \vert \vert {x}_{i} - {UDV}^{T} {β}_{k} \vert [ \begin{matrix}2 \\ 2\end{matrix} ] - \frac{1}{2} {β}_{k}^{T} {Λβ}_{k} \qquad ( 24 )$$

$${\sum }_{i = 1}^{N} {r}_{ik} [ - {( {UDV}^{T} )}^{T} ( {x}_{i} - {UDV}^{T} {β}_{k} 0 ] + {σ}_{k} {Λβ}_{k} = 0 \qquad ( 25 )$$

$${\sum }_{i = 1}^{N} {r}_{ik} {( {UDV}^{T} )}^{T} {x}_{i} = {\hat{σ}}_{k} {Λβ}_{k} + {\sum }_{i = 1}^{N} {r}_{ik} [ {( {UDV}^{T} )}^{T} ( {UDV}^{T} ) {β}_{k} ] \qquad ( 26 )$$

$${VDU}^{T} {X}^{T} {r}_{k} = [ {σ}_{k} Λ + ( {r}_{k}^{T} 1 ) {VD}^{2} {V}^{T} ] {β}_{k} \qquad ( 27 )$$

$${\hat{β}}_{k} = \frac{1}{[ {r}_{k}^{T} 1 ]} {( \frac{{\hat{σ}}_{k} Λ}{[ {r}_{k}^{T} 1 ]} + {VD}^{2} {V}^{T} )}^{- 1} {VDU}^{T} {X}^{T} {r}_{k} . \qquad ( 28 )$$

$$\begin{matrix}{\hat{β}}_{k} = \frac{1}{[ {r}_{k}^{T} 1 ]} {( \frac{{\hat{σ}}_{k} Λ}{[ {r}_{k}^{T} 1 ]} + {VD}^{2} {V}^{T} )}^{- 1} {( VD )}^{2} ( {D}^{- 2} {V}^{T} ) ( {VDU}^{T} ) {X}^{T} {r}_{k} \\ = \frac{1}{[ {r}_{k}^{T} 1 ]} {( \frac{{\hat{σ}}_{k} Λ}{[ {r}_{k}^{T} 1 ]} + {VD}^{2} {V}^{T} )}^{- 1} {( VD )}^{2} {D}^{- 1} {U}^{T} {X}^{T} {r}_{k} \\ = \frac{1}{[ {r}_{k}^{T} 1 ]} {( \frac{{\hat{σ}}_{k} {Λ VD}^{- 2}}{[ {r}_{k}^{T} 1 ]} + {VD}^{2} {V}^{T} {VD}^{- 2} )}^{- 1} {D}^{- 1} {U}^{T} {X}^{T} {r}_{k} .\end{matrix}$$

Thus, Λ can be removed from the inverse:

$$= \frac{1}{[ {r}_{k}^{T} 1 ]} ψ {VV}^{T} {{ψ}^{- 1} ( \frac{{\hat{σ}}_{k} Λ {VD}^{- 2}}{[ {r}_{k}^{T} 1 ]} + V )}^{- 1} {D}^{- 1} {U}^{T} {X}^{T} {r}_{k}$$

$$= \frac{1}{[ {r}_{k}^{T} 1 ]} ψ V {( \frac{{\hat{σ}}_{k} Λ {VD}^{- 2}}{[ {r}_{k}^{T} 1 ]} + {V}^{T} ψ V )}^{- 1} {D}^{- 1} {U}^{T} {X}^{T} {r}_{k} . \qquad ( 29 )$$

$${\hat{β}}_{k} = \frac{1}{[ {r}_{k}^{T} 1 ]} ψ V {( \frac{{\hat{σ}}_{k} Λ {VD}^{- 2}}{[ {r}_{k}^{T} 1 ]} + {V}^{T} ψ V )}^{- 1} {D}^{- 1} {U}^{T} {X}^{T} {r}_{k} . \qquad ( 30 )$$

This computation of \hat{β}ₖ avoids numerical instability.

### Target Gene Entropy

The model allows calculation of the entropy of each target gene using the conditional distribution of the latent indicator zᵢ. This is given by:

$$H ( {Z}_{i} ) = {\sum }_{k = 1}^{K} {r}_{ik} {log}_{2} ( {r}_{ik} ) , \qquad ( 31 )$$

where

$${r}_{ik} {=}^{△} p ( {z}_{i} = k \vert {x}_{i} , θ ) . \qquad ( 32 )$$

Since entropy is a measure of uncertainty, it was hypothesized that gene entropy, or uncertainty in assignment to a gene module, could be interpreted as a proxy for multiple module membership, and thus be used to unveil the elements of hidden crosstalk in cancer.

What is claimed is:

1. A method of constructing a gene module, comprising:
obtaining expression data;
identifying candidate regulator genes and target genes from the expression data; and
constructing gene modules with candidate regulator genes and target genes using a Gaussian mixture model.

2. The method of claim 1, wherein the Gaussian mixture model is combined with a norm regularization.

3. The method of claim 2, wherein Gaussian mixture model is defined as follows: \hat{β}=((τ^{T}1)G^{T}G+σΛ)⁻¹(G^{T}X^{T}τ), where G are the regulator genes and X are the target genes.

4. The method of claim 2 further comprising:
performing a biological experiment to validate a relationship of at least one regulator gene and its target gene.

5. The method of claim 4, wherein the biological experiment involves modulation of regulator gene activity.

6. The method of claim 4, wherein the biological experiment involves modulation of regulator gene expression.

7. The method of claim 2, wherein two sets of gene modules are constructed; the method further comprising:
identifying communities within each set of the two sets of gene modules; and
comparing the communities to identify a shared or a unique biological process between the two sets of gene modules.

8. The method of claim 7, wherein each identified community is defined by an average pairwise Jaccard Index between two sets of gene modules.

9. The method of claim 7, wherein the each identified community within the two sets of gene modules are functionally annotated.

10. The method of claim 9, wherein the annotation of each identified community is performed using gene set enrichment analysis.

11. The method of claim 7, wherein a first constructed gene module is derived from expression data of a medical disorder and a second constructed gene module is derived from expression data of a healthy control.

12. The method of claim 11 further comprising:
identifying a drug target within the medical disorder, wherein the drug target is regulator gene in a community that is unique to the constructed gene modules of the medical disorder.

13. The method of claim 12, wherein the unique community is related to a pathology of the medical disorder.

14. The method of claim 12 further comprising:
performing a preclinical assessment to assess one or more compounds for modulating a function of the drug target.

15. The method of claim 14, wherein the preclinical assessment involves contacting a biological sample with the one or more compounds, wherein the biological sample contains the drug target.

16. The method of claim 15, wherein the one or more compounds comprises at least one of: small molecules, biologics, or medicinals.

17. The method of claim 15, wherein the biological sample comprises at least one of: a biological cell, tissue, a lysate, or an isolated protein.

18. The method of claim 11, wherein the medical disorder is a cancer.

19. The method of claim 18, wherein the healthy control comprises a biological sample that is of the same tissue type of the cancer.

20. The method of claim 1, wherein the expression data is RNA sequencing data.
